(19) United States
(12) Patent Application Publication
(10) Pub. No.: US 2021/0115413 A1
(43) Pub. Date: Apr. 22, 2021

(54) METHODS OF GLYCOENGINEERING PROTEOGLYCANS WITH DISTINCT GLYCAN STRUCTURES

(71) Applicants: Massachusetts Institute of Technology, Cambridge, MA (US); Pfizer Inc., New York, NY (US)

(72) Inventors: Michelle Chang, Cambridge, MA (US); Leonid A. Gaydukov, Tewksbury, MA (US); Giyoung Jung, Cambridge, MA (US); Nevin M. Summers, Cambridge, MA (US); Timothy Kuan-Ta Lu, Cambridge, MA (US); Ron Weiss, Newton, MA (US); John Scarcelli, Wilmington, MA (US); Richard Cornell, Woburn, MA (US); Jeffrey Marshall, Weare, NH (US); Bruno Figueroa, Andover, MA (US); Wen Allen Tseng, Somerville, MA (US)

(73) Assignees: Massachusetts Institute of Technology, Cambridge, MA (US); Pfizer Inc., New York, MA (US)

(21) Appl. No.: 17/253,946

(22) PCT Filed: Jun. 20, 2019
(86) PCT No.: PCT/US2019/038217

§ 371 Date: Dec. 18, 2020

Related U.S. Application Data
(60) Provisional application No. 62/687,648, filed on Jun. 20, 2018.

(51) Int. Cl. C12N 9/10; C07K 16/00; C12N 15/90

(52) U.S. Cl. CPC C12N 9/1051; C12Y 204/01068; C07K 16/00; C12N 15/907; C12N 2510/00; C07K 2317/41; C12N 2800/80; C12N 2310/20; C07K 2317/14

(57) ABSTRACT

Disclosed herein are methods of generating proteoglycans with distinct glycan structures in engineered, non-naturally occurring eukaryotic cells. These methods make accessible a dynamic range of protein glycosylation. Compositions of engineered, non-naturally occurring cells capable of generating these proteoglycans are also disclosed herein.

### RELATED APPLICATION

This application claims priority under 35 U.S.C. § 119(e) to U.S. provisional patent application No. 62/687,648, filed Jun. 20, 2018, the entire contents of which are incorporated herein by reference.

### FIELD

Disclosed herein are methods of generating proteoglycans with distinct glycan structures in engineered, non-naturally occurring eukaryotic cells. These methods make accessible a dynamic range of protein glycosylation. Compositions of engineered, non-naturally occurring cells capable of generating these proteoglycans are also disclosed herein.

### BACKGROUND

Protein glycosylation can impact in vivo and in vitro structural and functional properties of therapeutic proteins, such as pharmacokinetic properties and potency. Monoclonal antibodies (mAbs) have been utilized for a wide variety of therapeutic applications, including the treatment of several cancers and autoimmune diseases (Weiner L. M., et al., Cell. 2012 Mar. 16; 148(6): 1081-84; Jefferis R., Trends Pharmacol. Sci. 2009 July; 30(7): 356-62; Chiu M. L. and Gilliland G. L., Curr. Opin. Struct. Biol. 2016 June; 38: 163-73). N-linked glycosylation significantly influences the structure, function, and pharmacokinetics of mAbs (Liu L., J. Pharm. Sci. 2015 June; 104(6): 1866-84). A high level of heterogeneity exists regarding N-linked glycosylation composition, branching and linkage position isomerization. As a result of this high level of heterogeneity, and the influence that these different glycoforms have on function, there has been increased interest in glycoengineering biopharmaceuticals to obtain products with distinct N-linked glycan structures. One strategy to manipulate N-linked glycosylation is based on in-process controls such as culture temperature, pH, and feed (Li F., et al., MAbs. 2010 September-October; 2(5): 466-79). Other options include the use of specific inhibitors or RNAi constructs to knock down glycosyltransferase activity or protein expression levels. Additionally, glycosyltransferase levels can be significantly reduced by silencing or removing the associated gene as well as removing the genes necessary for monosaccharide biosynthesis. Several examples have been demonstrated using this approach to generate afucosylated proteins (Kanda Y., et al., J. Biotechnol. 2007 Jun. 20; 130(3): 300-10; Yamane-Ohnuki N., et al., Biotechnol. Bioeng. 2004 Sep. 5; 87(5): 614-22; Mori K., et al., Biotechnol. Bioeng. 2004 Dec. 30; 88(7): 901-8; Yang Z., et al., Nat. Biotechnol. 2015 August; 33(8): 842-44).

### SUMMARY

Disclosed herein are methods of generating proteoglycans with distinct glycan structures in engineered, non-naturally occurring eukaryotic cells. This route of genome engineering makes accessible a dynamic range of protein glycosylation that has never been observed. Also disclosed herein are novel compositions of engineered, non-naturally occurring cells capable of generating these proteoglycans. Proteoglycans that can be generated by the disclosed cells and in accordance with the disclosed methods include any therapeutic protein that is glycosylated and expressed in host cells, such as glycoproteins, monoclonal antibodies, Fc fusion proteins, and other engineered proteins.

In some aspects, the disclosure provides engineered, non-naturally occurring eukaryotic cells including a modified genome, wherein the modified genome includes: (a) a knockout of at least one endogenous polynucleic acid sequence encoding a glycan modifying enzyme; and (b) an integration of at least one polynucleic acid sequence comprising a sequence encoding a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme.

In some embodiments, the tunable control element in (b) is selected from the group consisting of an inducible promoter element, a synthetic promoter panel, a miRNA response element, and an ORF control element.

In some embodiments, the engineered, non-naturally occurring eukaryotic cell comprises: (b) an integration of at least two polynucleic acid sequences, wherein each polynucleic acid sequence comprises the sequence of a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme. In some embodiments, the tunable control element of each of the at least two polynucleic acid sequences in (b) is unique. In some embodiments, the tunable control element of at least one of the at least two polynucleic acid sequences in (b) is selected from the group consisting of an inducible promoter element, a synthetic promoter panel, a miRNA response element, and an ORF control element.

In some embodiments of the engineered, non-naturally occurring eukaryotic cell, the tunable control element in (b) comprises an inducible promoter. In some embodiments, the inducible promotor is a chemically-regulated promoter or a physically-regulated promoter. In some embodiments, the chemically-regulated promoter comprises a TRE-Tight promoter sequence or a PhlF-activatable promoter sequence.

In some embodiments, the glycan modifying enzyme in (a) is selected from the group consisting of a fucosyltransferase, a galactosyltransferase, a sialyltransferase, an oligosaccharyltransferase, a glycosidase, a mannosidase, and a monoacylglycerol acetyltransferase. In some embodiments, the glycan modifying enzyme in (a) is FUT8. In some embodiments, the glycan modifying enzyme in (a) is β4GALT1. In some embodiments, the genome of the engineered, non-naturally occurring eukaryotic cell includes a knockout of at least two glycan modifying enzymes, wherein one of the at least two glycan modifying enzymes is FUT8 and one of the at least two glycan modifying enzymes is β4GALT1.

In some embodiments, the engineered, non-naturally occurring eukaryotic cell further comprises a polynucleic acid sequence comprising the sequence of a protein of interest operably linked to a constitutive or inducible promoter, wherein the protein of interest can be modified by the addition of a glycan. In some embodiments, the protein of interest is an immunoglobulin. In some embodiments, the immunoglobulin belongs to the IgA, IgD, IgE, IgG, or IgM class. In some embodiments, the immunoglobulin is an IgG1, IgG2, IgG3, or IgG4 immunoglobulin.

In some embodiments, the eukaryotic cell is a CHO cell, a COS cell, a NS0 cell, Sp2/0 cell, BHK cell, HEK293 cell, HEK293-EBNA1 cell, HEK293-F cell, HT-1080 cell, HKB-11, CAP cell, HuH-7 cell, or a PER.C6 cell. In some embodiments, the eukaryotic cell is a CHO cell.

In some embodiments, the integration of the at least one polynucleic acid sequence comprising the sequence of a functional copy of a glycan modifying enzyme and/or the integration of the at least one polynucleic acid sequence comprising the sequence of protein of interest operably linked to a constitutive or inducible promoter is at one or more landing pads.

According to another aspect, the disclosure provides methods of generating a glycoprotein including a distinct glycan structure. The methods include expressing at least one protein of interest and at least one glycan modifying enzyme in an engineered, non-naturally occurring eukaryotic cell including a modified genome, wherein the modified genome comprises: (a) a knockout of at least one endogenous polynucleic acid sequence encoding a glycan modifying enzyme; (b) an integration of at least one polynucleic acid sequence comprising a sequence encoding a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme; and (c) an integration of at least one polynucleic acid sequence comprising a sequence encoding a protein of interest operably linked to a constitutive or inducible promoter, wherein each of the at least one protein of interest can, when expressed as a protein, be modified by the addition of a glycan.

In some embodiments, the tunable control element in (b) is selected from the group consisting of an inducible promoter element, a synthetic promoter panel, a miRNA response element, and an ORF control element.

In some embodiments, the engineered, non-naturally occurring eukaryotic cell comprises: (b) an integration of at least two polynucleic acid sequences, wherein each polynucleic acid sequence comprises the sequence of a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme. In some embodiments, the tunable control element of each of the at least two polynucleic acid sequences in (b) is unique. In some embodiments, the tunable control element of at least one of the at least two polynucleic acid in (b) is selected from the group consisting of an inducible promoter element, a synthetic promoter panel, a miRNA response element, and an ORF control element.

In some embodiments, the tunable control element in (b) comprises an inducible promoter. In some embodiments, the inducible promotor is a chemically-regulated promoter or a physically-regulated promoter. In some embodiments, the chemically-regulated promoter includes a TRE-Tight promoter sequence or a PhlF-activatable promoter sequence.

In some embodiments, the glycan modifying enzymes in (a) is selected from the group consisting of a fucosyltransferase, a galactosyltransferase, a sialyltransferase, an oligosaccharyltransferase, a glycosidase, a mannosidase, and a monoacylglycerol acetyltransferase. In some embodiments, the glycan modifying enzymes in (a) is FUT8. In some embodiments, the glycan modifying enzymes in (a) is β4GALT1. In some embodiments, the genome of the engineered, non-naturally occurring eukaryotic cell comprises a knockout of at least two glycan modifying enzymes, wherein one of the at least two glycan modifying enzymes is FUT8 and one of the at least two glycan modifying enzymes is β4GALT1.

In some embodiments, the protein of interest of (c) is an immunoglobulin. In some embodiments, the immunoglobulin belongs to the IgA, IgD, IgE, IgG, or IgM class. In some embodiments, the immunoglobulin is an IgG1, IgG2, IgG3, or IgG4 immunoglobulin.

In some embodiments, the eukaryotic cell is a CHO cell, a COS cell, a NS0 cell, Sp2/0 cell, BHK cell, HEK293 cell, HEK293-EBNA1 cell, HEK293-F cell, HT-1080 cell, HKB-11, CAP cell, HuH-7 cell, or a PER.C6 cell. In some embodiments, the eukaryotic cell is a CHO cell.

In some embodiments, the integration of the at least one polynucleic acid sequence comprising the sequence of a functional copy of a glycan modifying enzyme and/or the integration of the at least one polynucleic acid sequence comprising the sequence of protein of interest operably linked to a constitutive or inducible promoter is at one or more landing pads.

According to another aspect, the disclosure provides an immunoglobulin generated by any of the methods disclosed herein. In some embodiments, the immunoglobulin includes at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% fucosylation. In some embodiments, the immunoglobulin includes at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% galactosylation. In some embodiments, the immunoglobulin includes at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% sialylation. In some embodiments, the immunoglobulin includes (a) at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% fucosylation; and/or (b) at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% galactosylation; and/or (c) at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% sialylation.

According to another aspect, the disclosure provides compositions including at least one immunoglobulin as disclosed herein.

These and other aspects of the invention are further described below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1C. Monoclonal antibody structure. FIG. 1A. Various regions and domains of a typical IgG with N-linked glycan attached at Asn297. FIG. 1B. Complex N-linked glycan structure with associated biological effects of each sugar. FIG. 1C. Denotation of commonly observed glycan structures, where G0, G1, and G2 indicate the number of terminal galactoses. F and S denote presence of fucose or sialic acid.

FIG. 2. Schematic diagram of landing pad donor vectors used for CRISPR/Cas9 targeted insertion into the LP2, Rosa, and C5 loci within the CHO genome. Key components include 5′ and 3′ locus-specific left and right homology arms (LHA and RHA), attP attachment site for BxB1 recombinase (wild-type or GA mutant), hEF1a constitutive promoter driving expression of a multicistronic gene consisting of an EBFP or EYFP fluorescent reporter protein and a selectable marker (blasticidin or hygromycin) fused together by the 2A self-cleaving peptide, and a termination sequence.

FIG. 3. Schematic map of 2×JUG-444 payload for integration into LP2 locus. 5′ attB attachment site for a wild-type BxB1 recombinase is necessary for DNA recombination with wild type BxB1 attP site of LP2. Puromycin (puro) resistance marker is used for selection of the integrated payload. Constitutive promoters mCMV and hEF1a drive expression of mAb light and heavy chains, respectively. Not shown are pairs of cHS4 insulators between each transcription unit.

FIG. 4. Schematic diagram of synthetic circuits for integration into Rosa locus of dLP cell lines. 5′ attB attachment site for a BxB1 (GA-mutant) (Inniss M. C., et al., Biotechnol. Bioeng. 2017 August; 114(8): 1837-46) recombinase is necessary for DNA recombination with BxB1 (GA-mutant) attP site of Rosa. Puromycin or blasticidin resistance marker is used for selection of the integrated payload. Not shown is pMC4.1, the Dox-inducible β4GALT1 circuit, with WT-attB-BxB1, which allows for the integration into C5 locus of tLP cell line.

FIG. 5. Genetic circuit for a weak constitutive expression of FUT8 with miRNA control. FUT8 gene is expressed from a weak constitutive promoter (hUBC or hACTB) and is flanked by miR-FF4 MRE sites at 5′ and 3′ regions, resulting in 1, 4 or 8 total MREs. In pMC17, miR-FF4 is constitutively expressed from hEF1a-mKate-intronic construct as a spliced-out intron. miR-FF4 binds to the complementary MREs on FUT8 mRNA, thus destabilizing FUT8 transcript. In pMC18 and pMC19 constructs, miR-FF4 is driven from a stronger U6 promoter to regulate FUT8 with 4 and 8 MREs, respectively.

FIG. 6. Genetic circuits for constitutive expression of FUT8 using a synthetic promoter library. Nearly 6000 different multiple TFBS are located upstream of a core promoter and they drive FUT8 expression. The synthetic promoter library provides wide range of FUT8 expression.

FIG. 7. Schematic diagram of genetic circuits with variable synthetic uORFs to tune translation levels to achieve lower FUT8 expression.

FIG. 8. Schematic diagram of synthetic circuits for integration into C5 locus of tLP cell lines. 5′ attB attachment site for a BxB1 (GA-mutant) (Inniss M. C., et al., Biotechnol. Bioeng. 2017 August; 114(8): 1837-46) recombinase is necessary for DNA recombination with BxB1 (GA-mutant) attP site of C5. Hygromycin resistance marker is used for selection of the integrated payload and EYFP is a fluorescent marker.

FIGS. 9A-9B. FUT8 and β4GALT1 knockouts. FIG. 9A. Exon excision with CRISPR/Cas9 and paired gRNAs confirmed by PCR of genomic DNA. FUT8 K/O is generated by excision of 5.2 kb. β4GALT1 K/O is generated by excision of 1.8 kb. FIG. 9B. HILIC analysis of JUG-444 released and labeled glycans from wild-type and generated knockout clones identified by PCR screen. Percentages of glycosylated species are indicated adjacent to or above the associated peak.

FIG. 10. HILIC glycan analysis of JUG-444 with pMC1 (encoding constitutively expressed FUT8) integrated in dLP FUT8 KO cell line and pMC2 (encoding constitutively expressed β4GALT1) integrated in dLP β4GALT1 KO cell line. Percentages of glycosylated species are indicated on above associated peak.

FIGS. 11A-11B. Plot of the relationship between percent fucosylation or galactosylation of JUG-444 and doxycycline resulting from the addition of doxycycline inducer (added every 48 hours) after 7-day fed-batch cultures. FIG. 11A. Percent total fucosylation levels when FUT8 expression is induced with variable Dox concentrations. FIG. 11B. Percent total galactosylation levels when β4GALT1 expression is induced with variable Dox concentrations.

FIGS. 12A-12B. Plot of the relationship between percent fucosylation or galactosylation of JUG-444 and abscisic acid resulting from the addition of abscisic acid inducer (added every 24 hours) after 7-day fed-batch cultures. FIG. 12A. Percent total fucosylation levels when FUT8 expression is induced with variable ABA concentrations. FIG. 12B. Percent total galactosylation levels when β4GALT1 expression is induced with variable ABA concentrations.

FIGS. 13A-13B. Plot of the relationship between percent fucosylation or galactosylation of JUG-444 and abscisic acid or doxycycline and abscisic acid resulting from the addition of small molecule inducers (ABA and Dox added every 24 and 48 hours, respectively) after 7-day fed-batch cultures. FIG. 13A. Percent total fucosylation levels when FUT8 expression is induced with variable ABA concentrations. Dox concentration is held constant at 1000 nM for all levels of ABA. FIG. 13B. Percent total galactosylation levels when β4GALT1 expression is induced with variable Dox concentrations. At 1000 nM Dox, total Gal levels were probed at several concentrations of ABA.

FIG. 14. FcγRIIIa binding SPR analysis of JUG-444 expressed with variable fucosylation and galactosylation levels. ABA (added every 24 h) and Dox (added every 48 h) concentrations used to induce the different glycosylation profiles are indicated. The bars indicate the K_{d} (nM) values of JUG-444 binding to the captured FcγRIIIa (158V) by SPR.

FIG. 15. Comparison of JUG-444 glycan composition in wild-type JUG-444 and in JUG-444 expressed in β4GALT1 KO cells expressing pMC2 and pMC20. G0, G1, and G2 indicate the number of terminal galactose residues. Bars are from left to right: G0, G1, G2, and Siaylated.

FIGS. 16A-16C. Constitutive FUT8 expression using promoter mini libraries. FIG. 16A. Schematic of FUT8 constitutively expressing circuits. Constitutive promoters were hEF1a, RSV, hPGK, hUBC, HSV-TK, and hACTB. FIG. 16B. FUT8 mRNA levels in cell lines containing FUT8 constitutively expressing circuits having the indicated constitutive promoter. FIG. 16C. Fucosylation levels of mAbs expressed in the cell lines of FIG. 16B.

FIGS. 17A-17C. Utilization of intronic miRNA circuits to control mAb N-glycan fucosylation. FIG. 17A. Schematic of FUT8 constitutively expressing circuits. To reduce the fucosylation level of mAb, miRNA targets (or binding sites (BS) in FIGS. 17B-17C) were inserted in the 3′ UTR of the synthetic FUT8 sequence. Constitutive promoters were RSV, hPGK, and hUBC. FIG. 17B. FUT8 mRNA levels in cell lines containing FUT8 constitutively expressing circuits having the indicated constitutive promoter. FIG. 17C. Fucosylation levels of mAbs expressed in the cell lines of FIG. 17B.

FIGS. 18A-18C. Utilization of U6 promoter-transcribed miRNAs circuits to control mAb N-glycan fucosylation. FIG. 18A. Schematic of FUT8 constitutively expressing circuits. To reduce the fucosylation level of mAb, miRNA targets (or binding sites (BS) in FIGS. 18B-18C) were inserted in the 3′ UTR and 5′ UTR of the synthetic FUT8 sequence. Constitutive promoters were hUBC and hACTB. FIG. 18B. FUT8 mRNA levels in cell lines containing FUT8 constitutively expressing circuits having the indicated constitutive promoter. FIG. 18C. Fucosylation levels of mAbs expressed in the cell lines of FIG. 18B.

FIGS. 19A-19D. Cell line stability of glycol-engineered cell lines. FIG. 19A. Fucosylation levels of mAbs expressed in MC1 cells analyzed at the indicated time. FIG. 19B. Fucosylation levels of mAbs expressed in GJ138 cells analyzed at the indicated time. FIG. 19C. Titer levels of mAbs expressed in MC1 cells analyzed at the indicated time. FIG. 19D. Titer levels of mAbs expressed in GJ138 cells analyzed at the indicated time.

### DETAILED DESCRIPTION

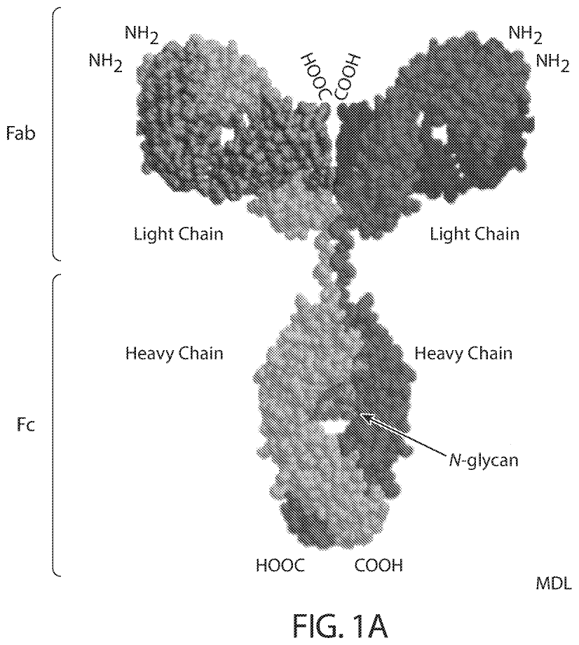

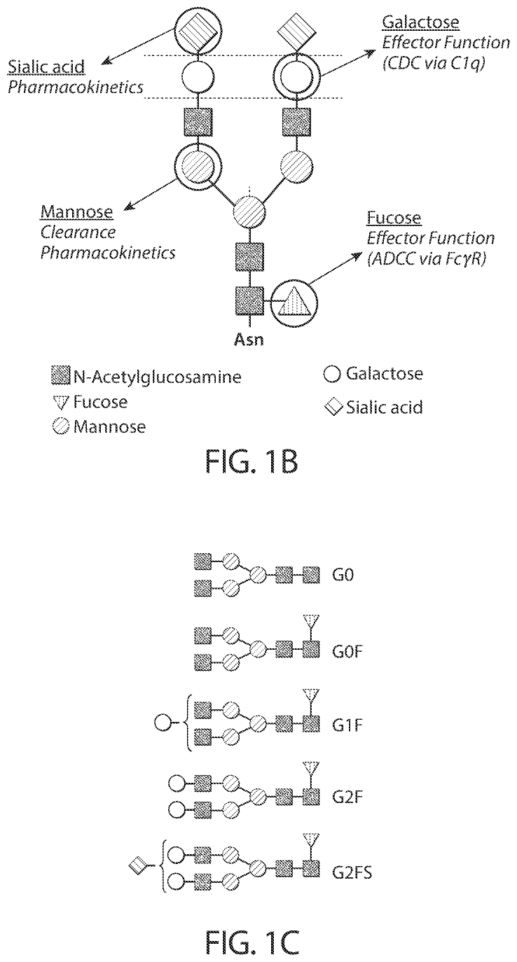

Therapeutic and engineered proteins that are produced by expression in mammalian cells, such as CHO cells, can have various properties altered by glycosylation, which can be influenced by the type of cell used, culture conditions, etc. One example of this is monoclonal antibodies (mAbs) which, have been utilized for a wide variety of therapeutic applications, including the treatment of several cancers and autoimmune diseases (Weiner L. M., et al., Cell. 2012 Mar. 16; 148(6): 1081-84; Jefferis R., Trends Pharmacol. Sci. 2009 July; 30(7): 356-62; Chiu M. L. and Gilliland G. L., Curr. Opin. Struct. Biol. 2016 June; 38: 163-73). All marketed mAbs belong to the IgG class and consist of two heavy chains and two light chains, with antigen-binding (Fab) and crystallizable (Fc) regions, where the Fc has the potential to bind to Fcγ receptors that regulate immune responses (FIG. 1A). Fc-mediated effector functions, such as antibody-dependent cell-mediated cytotoxicity (ADCC), antibody-dependent cellular phagocytosis (ADCP), and complement-dependent cytotoxicity (CDC), are important mechanisms of antibody therapies. N-linked glycosylation significantly influences the structure, function, and pharmacokinetics of mAbs (FIG. 1B) (Liu L., J. Pharm. Sci. 2015 June; 104(6): 1866-84). In the case of Fc glycosylation, N-acetylglucosamine (GlcNAc) is attached to Asn297 of the heavy chain and the glycan is subsequently processed in the ER and Golgi networks. N-linked glycans are very complex and diverse due to the high number of different sugar moieties and the multitude of possible linkages (FIG. 1C). As a result of this high level of heterogeneity, and the influence that these different glycoforms have on function, there has been increased interest in glycoengineering of biopharmaceuticals to obtain products with distinct N-linked glycan structures (Li F., et al., MAbs. 2010 September-October; 2(5): 466-79; Kanda Y., et al., J. Biotechnol. 2007 Jun. 20; 130(3): 300-10; Yamane-Ohnuki N., et al., Biotechnol. Bioeng. 2004 Sep. 5; 87(5): 614-22; Mori K., et al., Biotechnol. Bioeng. 2004 Dec. 30; 88(7): 901-8; Yang Z., et al., Nat. Biotechnol. 2015 August; 33(8): 842-44).

Disclosed herein are novel methods of glycoengineering proteoglycans with distinct glycan structures. The disclosed methods make accessible a dynamic range of protein glycosylation that has never been observed (e.g., 0-95% fucosylation and 0-85% total galactosylation of immunoglobulins). The disclosed methods provide precise, independent control of fucosylation and galactosylation that allows for a large matrix of Fc glycosylated species, which enables the development of new mAbs as well as other types of large molecule therapeutics with tailored in vitro and in vivo effects for use in biotechnology and biomedicine. Also demonstrated herein are the design and control of IgG glycoforms to influence Fc effector function. Importantly, the methods described herein can be applied beyond IgG1s to IgG2, IgG3, and other recombinant glycoproteins where glycans are known to have potential clinical impact such as half-life and effector function.

In some aspects, the disclosure relates to methods of generating glycoproteins comprising a distinct glycan structure in vivo. In some embodiments, the method comprises expressing at least one protein of interest and at least one glycan modifying enzyme in an engineered, non-naturally occurring eukaryotic cell comprising a modified genome (described below), wherein each of the at least one protein of interest can, when expressed, be modified by the addition of a glycan.

In other aspects, the disclosure relates to engineered, non-naturally occurring eukaryotic cells comprising a modified genome. As used herein, the term “modified genome” refers to a genome that has been altered so as to render the genome different from that which occurs in nature. In some embodiments, the modified genome comprises: (a) a knockout of at least one endogenous polynucleic acid sequence encoding for a glycan modifying enzyme; and (b) an integration of at least one polynucleic acid sequence comprising the sequence of a functional copy of a glycan modifying enzyme knocked out in (a) and an operably linked tunable control element that controls mRNA and/or protein expression of the glycan modifying enzyme.

The term “glycan,” as used herein, refers to a polysaccharide or a compound consisting of at least two monosaccharides linked glycosidically or through a glycosidic bond (i.e., a type of covalent bond that joins a saccharide to another group, which may or may not be another saccharide).

The term “glycan modifying enzyme” as used herein, refers to a protein that catalyzes the formation of or the removal of a glycosidic bond. Examples of glycan modifying enzymes are known to those having skill in the art and include, but are not limited to, oligosaccharyltransferases, glycosidases, mannosidases, monoacylglycerol acetyltransferases, fucosyltransferases (e.g., FUT1, FUT2, FUT3, FUT4, FUT5, FUT6, FUT7, FUT8, FUT9, FUT10, and FUT11), galactosyltransferases (e.g., β3GALNT1, β3GALNT2, β3GALT1, β3GALT2, β3GALT4, β3GALT5, β3GALT6, β3GNT2, β3GNT3, β3GNT4, β3GNT5, β3GNT6, β3GNT7, β3GNT8, β4GALNT1, β4GALNT2, β4GALNT3, βGALNT4, β4GALT1, β4GALT2, β4GALT3, β4GALT4, β4GALT5, β4GALT6, β4GALT7, GALNT1, GALNT2, GALNT3, GALNT4, GALNT5, GALNT6, GALNT7, GALNT8, GALNT9, GALNT10, GALNT11, GALNT12, GALNT13, GALNT14, GALNTL1, GALNTL2, GALNTL4, GALNTL5, and GALNTL6), and sialyltransferases (e.g., SIAT4C, SIAT9, ST3GAL1, ST3GAL2, ST3GAL3, ST3GAL4, ST3GAL5, ST3GAL6, ST3GalIII, ST6GAL1, ST6GAL2, ST6Gal, ST8SIA1, ST8SIA2, ST8SIA3, ST8SIA4, ST8SIA5, ST8SIA6, and ST8Sia).

As used herein, the term “knockout” refers to a disruption of an endogenous gene, such that the endogenous gene is rendered inactive. In some embodiments, a knockout is rendered through excision or removal of at least a portion of an endogenous polynucleic acid sequence encoding for a gene (i.e., at least a portion of a gene-coding region). As used herein the term “at least a portion of” may refer to a single nucleotide or to a stretch of contiguous nucleic acids comprising at least 0.5%, at least 1%, at least 5%, at least 10%, at least 20%, at least 30%, at least 40%, at least 50%, at least 60%, at least 70%, at least 80%, at least 90%, at least 95%, or 100% of the gene coding polynucleic acid sequence. In some embodiments, a knockout is rendered through integration or introduction of an exogenous piece of DNA. As used herein, the term “integration” refers to the insertion or knockin of an exogenous sequence of DNA into the genome of a cell. Methods of performing gene knockout and knockin are known to those having skill in the art and include, but are not limited to, the use of homologous recombination and site-specific nucleases (e.g., recombinases, zinc-finger nucleases, TALENs, and CRISPR/Cas).

In some embodiments, the one or more polynucleic acid sequences integrated into the cell are integrated at one or more “landing pads” (LPs), which are defined sites in the genome of the cell. As described elsewhere herein, a landing pad can contain a recombination site(s) for site-specific integration of one or more polynucleic acid sequences using a recombinase that recognizes the recombination site(s) and effects recombination. In addition, the landing pad can contain a selectable marker. In “multi-LP” cell lines, multiple landing pads are used, and preferably in such cases the landing pads are orthogonal.

In some embodiments, the modified genome comprises a knockout of more than one endogenous polynucleic acid sequence encoding for a glycan modifying enzyme. In some embodiments, the number of integrated polynucleic acid sequences that comprise the sequence of a functional copy of a knocked out glycan modifying enzyme and an operably linked tunable control element is less than the number of knocked out glycan modifying enzymes (e.g., a knockout of FUT8 and β4GALT1 and an integration of a functional copy of FUT8, and not β4GALT1, or vice versa).

The term “functional copy,” as used herein, relates to the degree of identity between a polynucleic acid encoding for a native protein (i.e., the sequence found in a native cell) and an in vitro created polynucleic acid encoding for an engineered protein (i.e., the functional copy). In some embodiments, the polynucleic acid sequence of the native protein and the polynucleic acid sequence encoding for the functional copy are identical. In other embodiments, the sequences differ. For example in some embodiments, the polynucleic acid sequence encoding for the native protein and the polynucleic acid sequence encoding for the functional copy share 5-10%, 10-20%, 20-30%, 30-40%, 40-50%, 50-60%, 60-70%, 70-80%, 80-90%, 90-95%, 95-99%, or 99-100% identity. In some embodiments, the polynucleic acid sequence encoding for the functional copy is longer or shorter than the polynucleic acid sequence of the native protein by at least 5, at least 10, at least 20, at least 50, at least 100, at least 200, at least 300, at least 500, at least 1000, or greater than 1000 nucleotides. Indeed, in some embodiments, the polynucleic acid sequence of the functional copy may encode for protein functional properties that are not shared with the native protein (e.g., the protein encoded by the functional property can perform at least one function that is not shared with the native protein). In other embodiments, the polynucleic acid sequence of the functional copy of the glycan modifying enzyme may not encode for at least one function of the native protein (e.g., the native protein can perform at least one function that is not shared with the functional copy). Nonetheless, to be considered a “functional copy,” the polynucleic acid encoding for the engineered protein must maintain enough identity with that of the native protein such that the engineered protein can perform the targeted function of the native protein to at least some degree, such as at least 1%, at least 2%, at least 5%, at least 10%, at least 20%, at least 30%, at least 40%, at least 50%, at least 60%, at least 70%, at least 80%, at least 90%, or at least 95% of the activity of the native protein. In some embodiments, the targeted function is the ability to catalyze the formation of or the removal of a glycosidic bond. For example, if the native protein is a glycan modifying enzyme, a functional copy of the glycan modifying enzyme (e.g., fucosyltransferase functional copy) will have enough identity with the native glycan modifying enzyme (e.g., native fucosyltransferase) such that the functional copy can catalyze the formation of or the removal of a glycosidic bond (e.g., transfer L-fucose from a GDP-fucose donor substrate to an acceptor substrate) at least 1%, at least 2%, at least 5%, at least 10%, at least 20%, at least 30%, at least 40%, at least 50%, at least 60%, at least 70%, at least 80%, at least 90%, or at least 95% as efficiently as the native glycan modifying enzyme. In some embodiments, the engineered protein encoded by the functional copy can perform the targeted function more efficiently than the native protein (e.g., at least 110%, at least 120%, at least 150%, at least 200%, at least 300%, or greater than 500% of the activity of the native protein).

As used herein, the term “tunable control element” refers to a polynucleic acid sequence that can be modified to increase or decrease a particular output. In some embodiments, the tunable control element functions to regulate mRNA expression (i.e., the output is mRNA levels). For example, in some embodiments, the tunable control element comprises an inducible promoter (see e.g., Materials and Methods, Design, Example 3 and Example 4), a synthetic promoter panel (see e.g., Design—“Transcriptional regulation of FUT8 by synthetic promoter library”), and/or a miRNA response element (see e.g., Design—“microRNA control of synthetic genes expression”). In some embodiments, the tunable control element functions to regulate protein expression (i.e., the output is protein level). For example, in some embodiments, a tunable control element comprises an open reading frame (ORF) control element (see e.g., Design—“Upstream ORF control of synthetic gene expression”). In some embodiments, the tunable control element functions to regulate protein function (i.e., the output is protein function). For example, in some embodiments, the tunable control element comprises a polynucleic acid sequence that encodes for a protein that increases or decreases the activity of the protein generating the output.

In some embodiments, the engineered, non-naturally occurring eukaryotic cell comprises an integration of at least two polynucleic acid sequences, wherein each comprises the sequence of a functional copy of a glycan modifying protein and an operably linked tunable control element that controls mRNA and/or protein expression of the glycan modifying enzyme. In some embodiments, the tunable control element of each of the at least two polynucleic acid sequences—each comprising the sequence of a functional copy of a glycan modifying enzyme and an operably linked tunable control element—is unique, i.e., different than all other tunable control elements that control mRNA and/or protein expression of glycan modifying enzymes in the engineered, non-naturally occurring eukaryotic cell.

A tunable control element controls expression or transcription of the polynucleic acid sequence to which it is operably linked, such as a polynucleic acid sequence encoding a functional copy of a knocked out glycan modifying enzyme. A tunable control element is considered to be “operably linked” when it is in a correct functional location and orientation in relation to the polynucleic acid sequence it regulates, thereby resulting in the ability of the tunable control element to control transcription initiation or expression of that polynucleic acid sequence.

In some embodiments, the tunable control element comprises an inducible promoter. In some embodiments, the inducible promotor is a chemically-regulated promoter or a physically-regulated promoter. Examples of chemically-regulated promoters are known to those having skill in the art and include, but are not limited to, alcohol-regulated promoters, tetracycline-regulated promoters, steroid-regulated promoters, metal-regulated promoters, and pathogenesis-related promoters. As such, chemically regulated promoters may be responsive to the presence of small molecule inducers (e.g., ABA, Dox, cumate, and gibberellic acid). In some embodiments, the chemically-regulated promoter comprises the polynucleic acid sequence of a TRE-Tight promoter or a PhlF-activatable promoter. Examples of physically-regulated promoters are also known to those having skill in the art and include, but are not limited to, temperature-regulated promoters and light-regulated promoters.

In some embodiments, at least one of the glycan modifying enzymes that is knocked out in the engineered, non-naturally occurring eukaryotic cell is selected from the group consisting of an oligosaccharyltransferase, a glycosidase, a mannosidase, a monoacylglycerol acetyltransferase, a fucosyltransferase, a galactosyltransferase, and a sialyltransferase.

In some embodiments, at least one of the glycan modifying enzymes that is knocked out in the engineered, non-naturally occurring eukaryotic cell is a fucosyltransferase selected from the group consisting of FUT1, FUT2, FUT3, FUT4, FUT5, FUT6, FUT7, FUT8, FUT9, FUT10, FUT11, and orthologs thereof. In some embodiments at least one of the glycan modifying enzymes is FUT8.

In some embodiments, at least one of the glycan modifying enzymes that is knocked out in the engineered, non-naturally occurring eukaryotic cell is a galactosyltransferase selected from the group consisting of β3GALNT1, β3GALNT2, β3GALT1, β3GALT2, β3GALT4, β3GALT5, β3GALT6, β3GNT2, β3GNT3, β3GNT4, β3GNT5, β3GNT6, β3GNT7, β3GNT8, β4GALNT1, β4GALNT2, β4GALNT3, β8GALNT4, B4GALT1, β4GALT2, β4GALT3, β4GALT4, β4GALT5, β4GALT6, β4GALT7, GALNT1, GALNT2, GALNT3, GALNT4, GALNT5, GALNT6, GALNT7, GALNT8, GALNT9, GALNT10, GALNT11, GALNT12, GALNT13, GALNT14, GALNTL1, GALNTL2, GALNTL4, GALNTL5, GALNTL6, and orthologs thereof). In some embodiments, at least one of the glycan modifying enzymes is β4GALT1.

In some embodiments, at least one of the glycan modifying enzymes that is knocked out in the engineered, non-naturally occurring eukaryotic cell is a sialyltransferase selected from the group consisting of SIAT4C, SIAT9, ST3GAL1, ST3GAL2, ST3GAL3, ST3GAL4, ST3GAL5, ST3GAL6, ST3GalIII, ST6GAL1, ST6GAL2, ST6Gal, ST8SIA1, ST8SIA2, ST8SIA3, ST8SIA4, ST8SIA5, ST8SIA6, ST8Sia, and orthologs thereof.

In some embodiments, the genome of the engineered, non-naturally occurring eukaryotic cell comprises a knockout of at least two glycan modifying enzymes, wherein one of the at least two glycan modifying enzymes is FUT8 and one of the at least two glycan modifying enzymes is β4GALT1.

In some embodiments, the engineered, non-naturally occurring eukaryotic cell further comprises an integration of at least one polynucleic acid sequence comprising a sequence encoding a protein of interest operably linked to a constitutive or inducible promoter, wherein the protein of interest can be modified by the addition of a glycan. In some embodiments, a polynucleic acid sequence encoding for a protein of interest operably linked to a constitutive or inducible promoter is integrated as multiple copies, potentially at multiple genomic locations. In some embodiments, the constitutive or inducible promoter of the multiple copies is identical. In other embodiments, the constitutive or inducible promoter of at least one copy is unique.

In some embodiments, at least one protein of interest is an immunoglobulin (see e.g., Materials and Methods, Design, and Examples 2-6). In some embodiments, the immunoglobulin belongs to the IgA, IgD, IgE, IgG, or IgM class. In some embodiments, the immunoglobulin is an IgG1, IgG2, IgG3, or IgG4 immunoglobulin.

Various eukaryotic cells have been used to generate glycan-modified proteins. See e.g., Lalonde M. E. and Duocher Y., J. Biotechnol. 2017 Jun. 10; 251: 128-140, the entirety of which is incorporated herein). In some embodiments, the engineered, non-naturally occurring eukaryotic cell is derived from a CHO cell, a COS cell, a NS0 cell, Sp2/0 cell, BHK cell, HEK293 cell, HEK293-EBNA1 cell, HEK293-F cell, HT-1080 cell, HKB-11, CAP cell, HuH-7 cell, or a PER.C6 cell. In some embodiments, the engineered, non-naturally occurring eukaryotic cell is derived from a CHO cell.

In other aspects, the disclosure relates to proteoglycans generated as described above. In some embodiments, the proteoglycan is an immunoglobulin. In some embodiments, the immunoglobulin comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% fucosylation. Methods of determining the percentage of fucosylation are known to those having skill in the art (see e.g., Materials and Methods). In some embodiments, the immunoglobulin comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% galactosylation. Methods of determining the percentage of galactosylation are known to those having skill in the art (see e.g., Materials and Methods). In some embodiments, the immunoglobulin comprises at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% sialylation. Methods of determining the percentage of sialylation are known to those having skill in the art (see e.g., Materials and Methods).

In some embodiments, the immunoglobulin comprises: (a) at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% fucosylation; (b) at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% galactosylation; and/or (c) at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% sialylation.

In some aspects, the disclosure relates to compositions comprising at least one proteoglycan. In some embodiments, the proteoglycan is an immunoglobulin. In some embodiments, the composition is a pharmaceutical composition, which may routinely contain pharmaceutically acceptable concentrations of salt, buffering agents, preservatives, compatible carriers, adjuvants, pharmaceutically acceptable excipients, and optionally other therapeutic ingredients. The nature of the pharmaceutical carrier, excipient, and other components of the pharmaceutical composition will depend on the mode of administration. The pharmaceutical compositions of the disclosure may be administered by any means and route known to the skilled artisan.

### EXAMPLES

### Example 1. Methods and Materials for Examples 2-8

CHO cell culture and transfections: Serum-free, suspension adapted CHO-K1 cells were grown in CD-CHO media, supplemented with 8 mM L-glutamine, at 37° C. and 7% CO₂ in flasks with shaking at 130 rpm. Seeding density was 3×10⁵ cells/mL, and cultures were split every 3 or 4 days. Transfections were always carried out using Neon electroporation (1600 V, 10 ms, 3 pulses) with 3×10⁵ cells per 10 ul transfection.

Generation of knockout cell lines and genomic PCR diagnostic test: Transfection of 250 ng U6-gRNA pairs and 250 ng pSP-Cas9(BB)-2A-GFP into dLP cells with JUG-444 integrated into LP2. Three days post transfection, GFP-positive single cells were FACS sorted and genomic DNA was assayed for exon excision.

TABLE 1
Sequences used in this study.

| | | | |
|---|---|---|---|
| | SEQ | | |
| | ID | | |
| Name | NO | Sequence | Notes |

gRNA (PAM) sequences - GeneArt CRISPR String
DNA (Thermo)

| | | |
|---|---|---|
| Fut8-gRNA2 | 1 | TTATTTGCTTGACATACACA (GGG) |
| Fut8-gRNA3 | 2 | GTAATCCTAGTGCTATAGTG (GGG) |
| B4GalT1- | 3 | ATTGCAACAGAAATGTGCCG (GGG) |
| gRNA2 | | |
| B4GalT1- | 4 | TAGTGAGTCAGACCAAGACG (GGG) |
| gRNA4 | | |

PCR primers for gDNA PCR diagnostic test #1

| | | | |
|---|---|---|---|
| Fut8- | 5 | 5′-GAAAGATGGATTGACAGGGAGAG | 5726 bp |
| gRNA2-Fwd3 | | GTTAAG-3′ | amplicon |
| Fut8- | 6 | 5′-CAGGTGATGGGAGGGTTTTGATG | if no |
| gRNA3-Rev4 | | ATTTTC-3′ | excision. |
| | | | 499 bp |
| | | | amplicon |
| | | | if |
| | | | excision. |
| B4GalT1- | 7 | 5′-CTGGAAATGGATTGTTGACTCAG | 2428 bp |
| gRNA2-Fwd3 | | AGGG-3′ | amplicon |
| | | | if no |
| B4GalT1- | 8 | 5′-GAGAACCATCACATAAACTAAGG | excision. |
| gRNA4-Rev2 | | AAAACACC-3′ | 636 bp |
| | | | amplicon |
| | | | if |
| | | | excision. |

PCR primers for gDNA PCR diagnostic test #2

| | | | |
|---|---|---|---|
| Fut8- | 9 | 5′-CTTCCCTTTGACTCCACTTCTAT | 499 bp |
| gRNA3-Fwd3 | | GAAATTG-3′ | amplicon |
| Fut8- | 10 | 5′-CAGGTGATGGGAGGGTTTTGATG | if no |
| gRNA3-Rev4 | | ATTTTC-3′ | excision. |
| | | | No ampli- |
| | | | con if |
| | | | excision. |
| B4GalT1- | 11 | 5′-GTTTGTACTCTGACCCTTCTTAT | 924 bp |
| gRNA3-Fwd3 | | TCCTCTC-3′ | amplicon |
| B4GalT1- | 12 | 5′-GAGAACCATCACATAAACTAAGG | if no |
| gRNA4-Rev2 | | AAAACACC-3′ | excision. |
| | | | No ampli- |
| | | | con if |
| | | | excision. |

RT-qPCR primers

| | | |
|---|---|---|
| fut8-ex7- | 13 | 5′-ACTGGAGGATGGGAGACTGTGT- |
| fwd2 | | 3′ |
| fut8-ex8- | 14 | 5′-TCAGGAGTCGATCTGCAAGGTC |
| rev2 | | T-3′ |
| b4galt1- | 15 | 5′-TCTGTTGCAATGGACAAGTTTG |
| ex2-fwd4 | | G-3′ |
| b4galt1- | 16 | 5′-CCTCCCCAGCCCCAATAATTAT |
| ex3-rev3 | | T-3′ |

Construction and integration of genetic circuits: A synthetic FUT8 gene (cDNA sequence comprising 11 exons) and a synthetic β4GALT1 gene (cDNA sequence comprising 5 exons) were acquired as a gBlock from IDT. Modular Gateway/Gibson assembly was used in the construction of all genetic circuits (Duportet X., et al., Nucleic Acids Res. 2014 Dec. 1; 42(21): 13440-51). Circuit integration requires transfection of 500 ng pEXPR-BxB1 and at least 500 ng of each circuit. Three days post transfection, mKate signal was assayed by FACS analysis. Selection may be carried out for 7 days. Ten days post transfection, cells were sorted by FACS to obtain mKate-positive and EYFP-negative cells (circuit integration into Rosa locus) or for mKate-positive and EBFP-negative cells (circuit integration into C5 locus).

Fed-batch culture and glycan analysis: 7-day fed batch cultures were used to generate mAb for glycan analysis. Fed batch cultures (25 mL in 125 mL shake flasks) were seeded at 1.5×10⁶ cells/mL. Starting on day 3, cultures were titrated to pH 7.2 twice a day and supplemented with Cell Boost 5, 20% D-glucose, and L-glutamine once a day. When required to induce synthetic circuits, Dox was added every 48 hours or ABA was added every 24 hours to the fed batch culture starting on day 0. Cultures were harvested on day 7 and clarified media was saved for titer measurement by Octet and for JUG-444 purification on ProA resin. Glycans were enzymatically cleaved off of purified JUG-444, derivatized with 2-aminobenzamide labeling agent, and analyzed by HILIC (Shang T. Q., et al., J. Pharm. Sci. 2014 July; 103(7): 1967-78).

### FcγRIIIa Binding SPR Analysis:

Equipment and software: Biacore™ T200 instrument (GE Healthcare) with Control Software version 2.0.1 and Evaluation software version 3.0 was used for interaction analysis.

Sensor chips, reagents and buffers: Amine coupling reagents, N-(3-dimethylaminopropyl)-N-ethylcarbodiimide (EDC) and N-hydroxysuccinimide (NHS), ethanolamine-HCl, Series S Sensor Chip CMS, including 10 mM Glycine pH 1.5 regeneration solution, 10 mM Sodium Acetate pH 4.5, 50 mM Sodium Hydroxide, Biacore Normalizing Solution (70% Glycerol), 0.5% (w/v) sodium dodecyl sulphate, 50 mM Glycine pH 9.5 and HBS-EP+ Buffer 10× (0.1 M HEPES buffer with 30 mM EDTA, 1.5 M NaCl and 0.5% Surfactant P20 (Tween 20)) were purchased from GE Healthcare. Recombinant human FcγRIIIa-158V (ligand) expressed in Human Embyronic Kidney 293 (HEK293) cells was from Syngene and anti-PENTA Histidine antibody was from Qiagen. The JUG-444 mAbs used in this study were fully human mAbs expressed with IgG1. All JUG-444 mAbs were purified in-house and were later dialyzed into PBS. Finally, all the purified mAbs were aliquoted and stored at 10° C. until used for kinetic assay.

Immobilization of anti-PENTA Histidine mAb on Biacore T200: Anti-PENTA Histidine mAb diluted in 10 mM sodium acetate (pH 4.5) at 10 μg/ml was directly immobilized across a Series S CMS biosensor chip using a standard amine coupling kit according to manufacturer's instructions and procedures. Un-reacted moieties on the biosensor surface were blocked with ethanolamine. Anti-PENTA Histidine mAb immobilization procedure yielded approximately 1500 RU surface density. Modified carboxymethyl dextran surface containing captured Fcγ receptor via immobilized anti-PENTA Histidine Mab across flow cells 2 and 4 were used as a reaction surface. A similar modified carboxymethyl dextran surface without Fcγ receptors across flow cells 1 and 3 were used as a reference surface.

FcγRIIIa-158V capture assay procedure: The sample compartment of the Biacore T200 system was set to 10° C., the analysis temperature to 25° C. and the data collection rate to 1 Hz. HBS-EP+ was used as running buffer. In each cycle FcγRIIIa-158V (ligand) at 1 μg/ml in HBS-EP+ was injected for 60 seconds at a flow rate of 50 μl/min, to reach minimum capture levels of around 30-60 RU. JUG-444 antibody, 4.7 to 150.4 μg/ml in HBS-EP+, was injected for 180 seconds followed by a dissociation phase of 300 s for all six antigen concentrations and the surface was regenerated with 10 mM Glycine pH 1.5 solution per kit instructions (300 s contact). The association and dissociation rate constants, kₐ (unit M⁻¹s⁻¹) and k_{d} (unit s⁻¹) were determined under a continuous flow rate of 50 μl/min.

Data processing and analysis: The binding data were initially processed using the Evaluation version 3.0 software. The double reference subtracted data generated using FcγRIIIa-158V capture assay was globally fitted to a 1:1 Langmuir binding model. Rate constants for the JUG-444 mAb-FcγRIIIa-158V interactions were derived by making kinetic binding measurements at six different analyte concentrations ranging from 31.25-1000 nM. Association and dissociation rate constants were extracted from binding data using global fit analysis (allowing identical values for each curve in the data set). The Rₘₐₓ parameter setting was floated fit locally. The equilibrium dissociation constant (unit M) of the reaction between Fcγ receptor and JUG-444 mAbs was then calculated from the kinetic rate constants by the following formula: K_{D}=k_{d}/kₐ.

### Example 2. Design

Overview: CHO-K1 cells adapted for serum-free and suspension culture were used to construct new cell lines with knockouts of FUT8 and/or β4GALT1 genes, and with multiple landing pads for specific integration of JUG-444 and synthetic gene circuits. First, a landing pad (LP) containing a recombination site and a selectable marker was integrated into the genome. Then, a matching recombinase was used to insert a DNA payload specifically into that locus, allowing for reproducible integration at well-defined sites in the genome. By utilizing landing pads for both the mAb and the gene circuits, the cells were normalized for both copy number and loci, allowing for consistent and reproducible expression levels (Duportet X., et al., Nucleic Acids Res. 2014 Dec. 1; 42(21): 13440-51; Gaidukov L., et al., Nucleic Acids Res. 2018 May 4; 46(8): 4072-86). JUG-444 is an antibody of the IgG1 subclass, and the glycosylation of JUG-444 served as the functional readout for the modulation of Fut8 and β4GalT1 enzymatic activity. Synthetic circuits integrated into landing pads reintroduced FUT8 and β4GALT1 genes under constitutive or inducible promoters. Upon addition of small molecule inducers, varied levels of Fut8 and β4GalT1 enzymes were expressed corresponding to levels of small molecules added. This in turn led to varied levels of JUG-444 glycosylation that reflect the expressed enzyme levels. While JUG-444 was used as a test mAb, this system is compatible with all types of mAbs, including antibody-drug conjugates and bispecific monoclonal antibodies. In fact, it is relevant to any bio-manufactured genetically expressed therapeutic protein where precision in glycosylation is required for desired biological effect.

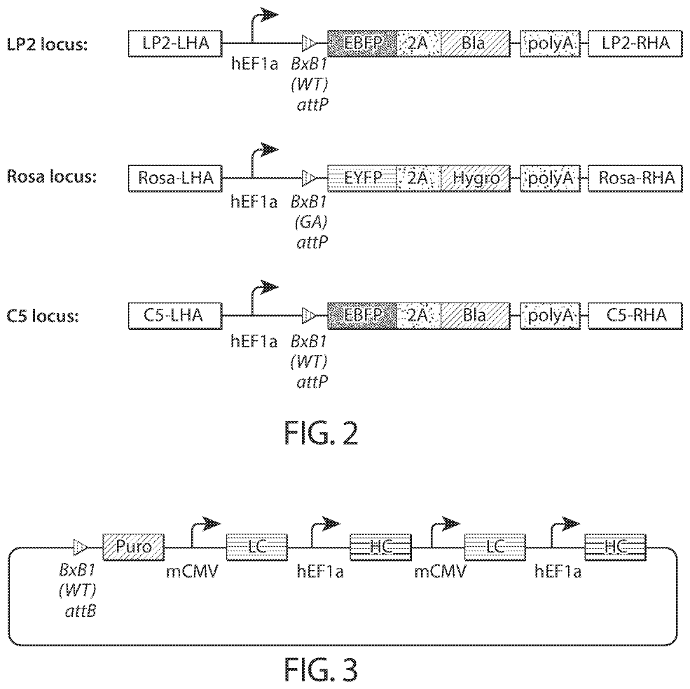

Generation of CHO cell lines with orthogonal landing pads: A landing pad (LP) containing a recombination site and a selectable marker was integrated using a CRISPR/Cas9 genome editing approach at loci demonstrated to have stable gene expression (Duportet X., et al., Nucleic Acids Res. 2014 Dec. 1; 42(21): 13440-51; Gaidukov L., et al., Nucleic Acids Res. 2018 May 4; 46(8): 4072-86) (FIG. 2). A matching site-specific recombinase is then used to insert a DNA payload specifically into that locus. Two or three orthogonal recombination sites with different fluorescent reporters and antibiotic selection markers are used to target payload integration into specific landing pad sites in multi-LP cell lines. For the double landing pad (dLP) cell line, LP2 site is integrated with JUG-444 payload encoding two copies of heavy and light chain genes, and Rosa site is available for integration of synthetic circuits. For the triple landing pad (tLP) cell line, C5 landing pad is additionally available for integration of synthetic circuits.

Integration of mAb into landing pad: A double copy of JUG-444 light and heavy chain genes was integrated into the first landing pad (LP2). BxB1-mediated recombination occurred between LP2's attP site and the payload's attB site (FIG. 3). Given that the optimal light chain to heavy chain ratio by Western analysis is 3:1 for the highest mAb production in CHO-DG44 cells (Ho S. C., et al., J. Biotechnol. 2013 Jun. 10; 165(3-4): 157-66), the LC was expressed from the stronger mCMV promoter and the HC was expressed from a weaker hEF1a promoter. In CHO-K1 cells, this configuration produced higher titers than one in which hEF1a is the promoter driving both LC and HC expression. This configuration should work for a wide range of protein expression.

Design of FUT8 and β4GALT1 knockouts: The CHO cell line with LP2 expressing JUG-444 was used to generate FUT8 and β4GALT1 knockouts by CRISPR/Cas9 targeted excision of exons within the catalytic domains of the glycosyltransferases. Exon 7 was completely excised from FUT8, and exon 2 was partially excised from β4GALT1. Functional knockouts were validated by analysis of JUG-444 glycan structures for lack of fucosylated or galactosylated species.

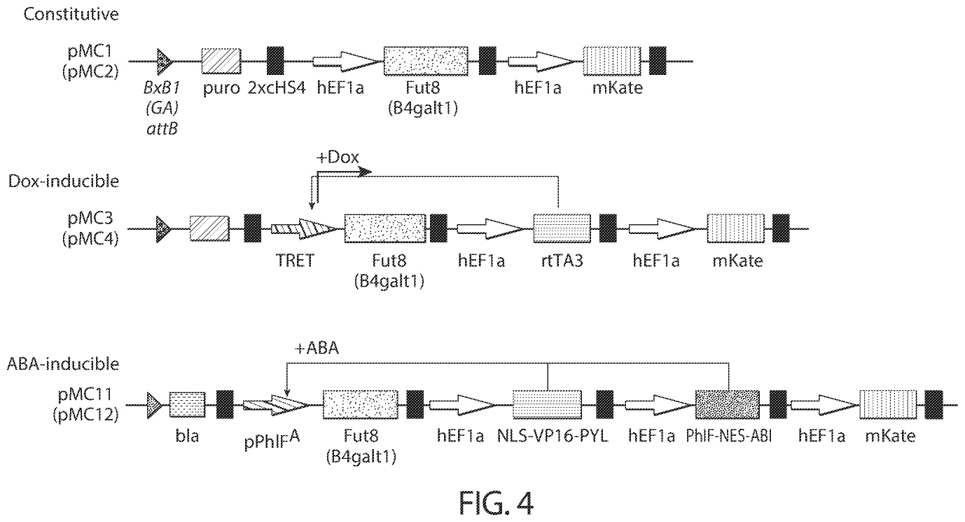

Design of FUT8 and β4 GALT1 synthetic circuits for integration into landing pads: Synthetic biological circuits were designed and constructed from an array of tunable and characterized parts, or modules, to perform logical functions that control cellular activities. In this example, synthetic FUT8 and β4 GALT1 genes were expressed under constitutive or small molecule inducible promoters (FIG. 4). All circuits also constitutively expressed mKate as a fluorescent marker. The constitutive promoter used was hEF1a for both FUT8 and β4 GALT1. In the Tet-On rtTA3 (reverse tetracycline transactivator) system, rtTA3 binds TRE-Tight promoter in the presence of doxycycline (Dox) and induces gene expression (Dow L. E., et al., PLoS One. 2014 Apr. 17; 9(4): e95236). In the abscisic acid (ABA)-induced dimerization system, a nuclear export signal (NES) on the PhlF DNA-binding domain and a nuclear localization signal (NLS) on VP16 transcription-activator domain sequester these ABI and PYL domains into different cellular compartments (Liang F. S., et al., Sci. Signal. 2011 Mar. 15; 4(164): rs2). PhlF and VP16 are also fused to ABI and PYL domains, respectively, that undergo dimerization in the presence of ABA to drive expression from a PhlF-activatable promoter. Circuits with synthetic FUT8 (pMC1, pMC3, and pMC11) were integrated into a dLP cell line expressing JUG-444 and having endogenous FUT8 knocked out. Circuits with synthetic β4 GALT1 (pMC2, pMC4, and pMC12) were integrated into a dLP cell line expressing JUG-444 and having endogenous β4 GALT1 knocked out. For simultaneous, independent control of fucosylation and galactosylation, pMC11 and pMC4.1 circuits were integrated into a tLP cell line expressing JUG-444 and having both endogenous FUT8 and β4 GALT1 knocked out.

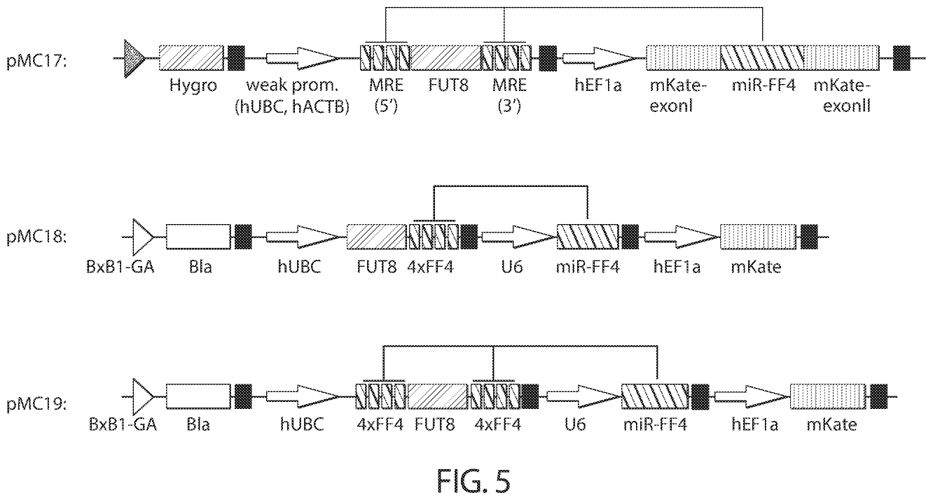

microRNA control of synthetic genes expression: MicroRNAs (miRNAs) are important elements of the RNA interference system that controls gene regulation in eukaryotic cells (Bartel D. P., Cell. 2009 Jan. 23; 136(2): 215-33). miRNAs are processed by protein complexes to knock down mRNA levels in the cell, reducing protein expression. As such, incorporating synthetic microRNA Response Elements (MREs) in the 5′- and/or 3′-UTR of a protein of interest can be used to further down-regulate already low level constitutive promoters whose gene expression levels need to be tuned down even further. Specifically, 1×, 4× and 8×MREs are incorporated for a particular miRNA (FF4) (e.g., to FUT8 synthetic gene that is expressed from a weak constitutive promoter (e.g., hUBC and hACTB)). The complimentary synthetic miRNA-FF4 is constitutively expressed from either a human U6 promoter (high miR-FF4 expression) or from hEF1a-mKate-intronic construct (low miR-FF4 expression). In the latter case miR-FF4 is produced as a spliced-out intron from the fluorescent protein mKate, and red fluorescence indicates the presence of miR-FF4 (FIG. 5). Combining different weak constitutive promoters with different number of MREs and different expression levels of miR-FF4 results in a library of weak constitutive promoters for various levels of FUT8 expression.

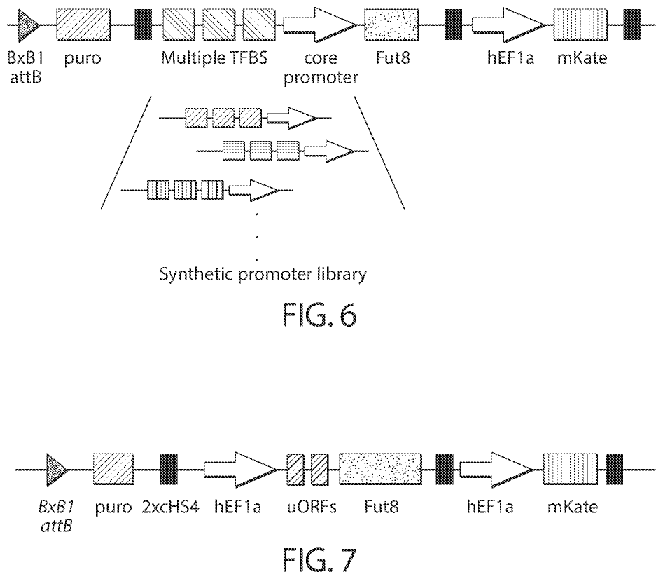

Transcriptional regulation of FUT8 by synthetic promoter library: A synthetic promoter library is another approach to fine-tune regulation of gene expression at the transcriptional level. Gene expression of FUT8 with commonly used constitutive promoters such as hEF1a resulted in very high expression of FUT8, leading to wild-type levels of antibody fucosylation. Even low FUT8 mRNA levels lead to high levels of fucosylation. Thus, to achieve a broad range of fucosylation, a weak and wide range of expression is required. A synthetic promoter library previously constructed (Nissim L., Cell. 2017 Nov. 16; 171(5): 1138-50), comprising nearly 6000 different multiple transcription factor binding sites (TFBS), provides a solution to this problem (FIG. 6). The strength of the synthetic promoter can be screened by the expression of a fluorescence marker in CHO cells. Multiple TFBS previously identified (Wingender E., et al., Nucleic Acids Res. 2013 January; 41; 24; Vaquerizas J. M., et al., Nat. Rev. Genet. 2009 April; 10(4): 252-63), are located upstream of the core promoter and FUT8 is expressed under this synthetic promoter.

Upstream ORF control of synthetic gene expression: Short, upstream open reading frames (uORFs), which encode a two-amino acid peptide, can be inserted upstream of an ORF encoding a protein of interest to suppress its expression (Ferreira J. P., et al., Proc. Natl. Acad. Sci. U.S.A. 2013 Jun. 9; 110(28): 11284-89). Varying the base sequence preceding the uORF or using multiple uORFs in series and non-AUG start codons results in variable translation initiation rates leading to expression levels spanning three orders of magnitude (FIG. 7). For example, FUT8 translation initiation can be controlled in this manner in order to tune FUT8 expression levels and achieve low levels of fucosylation in CHO cells.

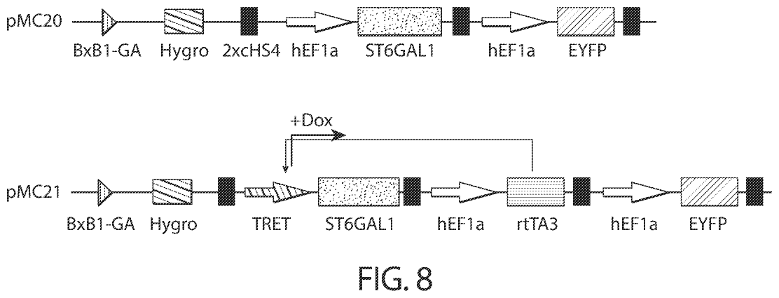

Design of synthetic circuits for tunable sialylation: Sialylation occurs on terminally galactosylated species and plays a role in anti-inflammatory activity of IgGs (Kaneko Y., Science. 2006 Aug. 4; 313(5787): 670-73). Galactosylation levels must be increased, as described in Example 2, before sialylation levels can be modulated. Cells expressing pMC2 in β4GALT1 KO cells can be used for integration of ST6GAL1 circuits in the third landing pad. Circuits with ST6GAL1 under constitutive and Dox-inducible promoters are used to modulate α-2,6-sialylation of JUG-444 (FIG. 8). If simultaneous and independent modulation of fucosylation, galactosylation, and sialylation is desired, another small molecule inducible system is necessary in addition to the ABA and Dox systems. Cumate (Mullick A., Xu Y., et al., BMC Biotechnol. 2006 Nov. 3; 6: 43) and gibberellic acid (Gao Y., et al., Nat. Methods. 2016 December; 13(12): 1043-49) inducible systems can be used as a third orthogonal inducible system.

### Example 3: Validation of FUT8 and β4GALT1 Knockouts

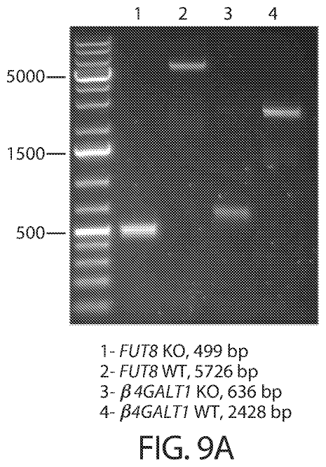

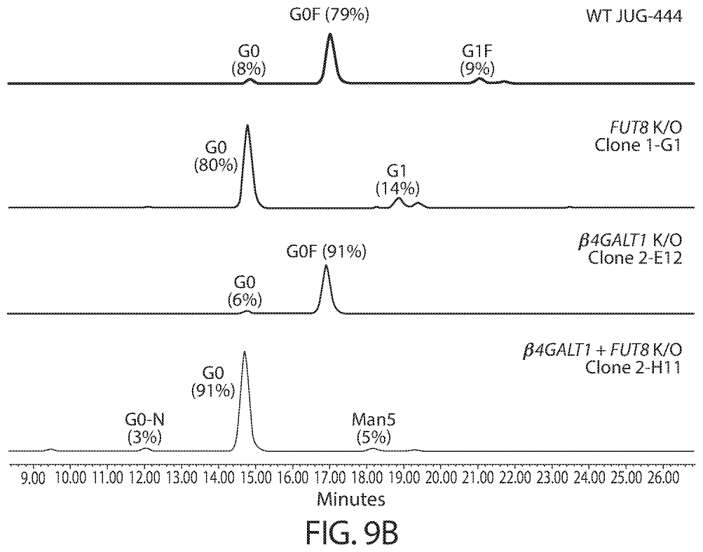

FUT8 and β4GALT1 knockouts (KO) were generated by CRISPR/Cas9 targeted excision of exons essential for catalytic activity, similar to what has been done previously (Zong H., et al., Eng. Life Sci. 2017 Feb. 23; 17(7): 801-8; Sun T., et al., Eng. Life Sci. 2015 Jul. 21; 15(6): 660-66). KO clones were identified with a PCR screen of genomic DNA (FIG. 9A). Enzymatically released and labeled JUG-444 glycans from each putative knockout were analyzed by hydrophobic interaction liquid chromatography (HILIC) and confirmed for the loss of fucosylated and/or galactosylated species (FIG. 9B). As expected, when compared to JUG-444 expressed in wild-type cells (dLP with no knockouts), the FUT8 KO clone exhibited conversion of G1F species to G1 and G0F species to G0. The β4GALT1 KO clone exhibited conversion of G1F species to G0F. The FUT8 and β4GALT1 double KO clone exhibited conversion of G0F and G1F species to G0, with increases in Man5 and G0-N species also observed.

### Example 4: FUT8 and β4GALT1 Expression Under Constitutive Promoters

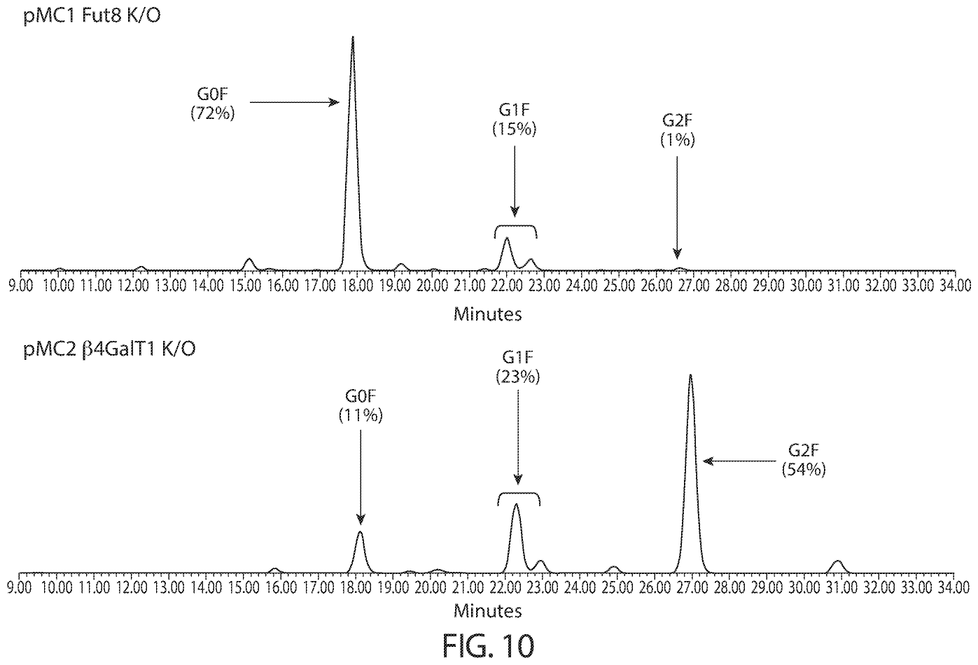

Integration of the pMC1 circuit (see FIG. 4) into the FUT8 KO cell line restored total fucosylation of JUG-444 to near wild-type levels at 92.7% (FIG. 10, TABLE 2). There was also a small increase in the levels of galactosylated species observed. Integration of pMC2 circuit (hEF1a promoter driving β4GALT1 expression) into the β4GALT1 KO cell line resulted in 87.1% total galactosylated species, which is a large increase from the 6.9% total galactosylated species found in B4GALT1 wild-type cells expressing JUG-444. Due to the significant increase in the levels of galactosylation reached, the percentage of sialylated species increased from 0.3% to 6.8%. Terminal galactosylation is a prerequisite for sialylation, so it is not surprising that the higher galactosylation levels resulted in higher levels of sialylation. The fucosylation and galactosylation levels observed with the relatively strong constitutive promoter hEF1a show that high fucosylation and galactosylation levels should be achievable with inducible systems, while the knockouts show the lowest levels of fucosylation and galactosylation that can be reached. An obvious extension of this work is to use weaker constitutive promoters, or weakened version of the hEF1a promoter, to achieve varying glycosylation levels.

TABLE 2
Composition of JUG-444 glycan species for FUT8 and β4GALT1 knockouts
and with integrated constitutive circuits, pMC1 and pMC2, where terminal
galactosylation refers to glycoforms with galactose terminating a glycan
branch, and total galactosylation refers to all glycoforms containing
galactose. Values marked with an asterisk include co-migration of other
low-level non-terminal galactosylated glycan species.

| | % | % High | % | % Terminal | % Total |
|---|---|---|---|---|---|
| Sample Name | Fucosylated | Mannose | Sialylated | Galactosylated | Galactosylated |
| WT JUG-444 | 91.7 | 2.8 | 0.3 | 6.6 | 6.9 |
| FUT8 KO | 0.0 | 0.9 | 0.2 | 14.0 | 14.2 |
| β4GALT1 KO | 90.8 | 1.6 | 0.3 | 2.3* | 2.6* |
| pMC1, FUT8 KO | 92.7 | 2.4 | 0.5 | 17.3 | 17.8 |
| pMC2, β4GALT1 KO | 93.5 | 0.8 | 6.8 | 80.3 | 87.1 |

### Example 5: FUT8 or β4GALT1 Single Gene Regulation Under Inducible Promoters

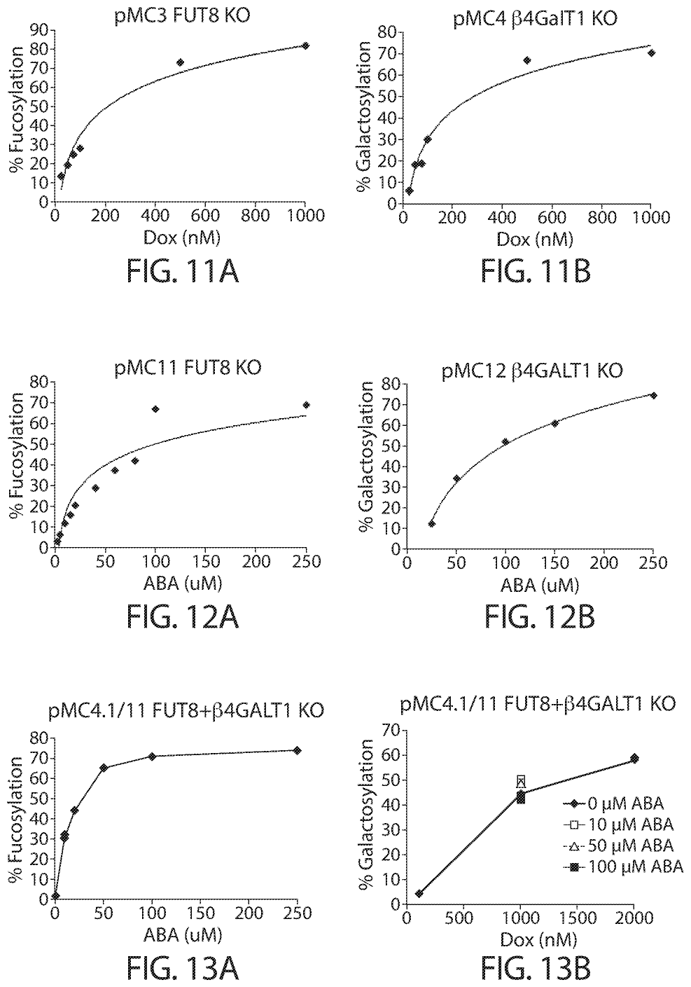

Dox-inducible circuits for regulating FUT8 and β4GALT1 expression (pMC3 and pMC4) were integrated into the FUT8 or β4GALT1 single knockout cell lines. With no addition of Dox, basal level of FUT8 expression due to leaky expression from TRET promoter from pMC3 resulted in 8.2% total fucosylation of JUG-444. The highest level of total fucosylation reached was 81.9% with 1000 nM Dox (FIG. 11A). The range from 8.2% to 81.9% is achievable through titration of Dox. Basal level of β4GALT1 expression from pMC4 resulted in 5.3% total galactosylation of JUG-444. The highest level of total galactosylation reached was 70.5% with 1000 nM Dox (FIG. 11B). The maximum fucosylation and galactosylation levels achieved in these experiments were a little lower than those seen with the hEF1a promoter. However, an altered inducer regimen during the 7-day fed batch cultures could be used to potentially achieve higher levels. It should be noted that expression levels from these landing pads remain robust and stable over a period of at least two months (Gaidukov L., et al., Nucleic Acids Res. 2018 May 4; 46(8): 4072-86).

ABA-inducible circuits for regulating FUT8 and β4GALT1 expression (pMC11 and pMC12) were integrated into the FUT8 or β4GALT1 single knockout cell lines. With no addition of ABA, basal level of FUT8 expression from pMC11 resulted in 2.0% total fucosylation of JUG-444. The highest level of total fucosylation reached was 68.8% with 250 uM ABA (FIG. 12A). The range from 2.0% to 68.8% is achievable through titration of ABA. Basal level of β4GALT1 expression from pMC12 resulted in 2.2% total galactosylation of JUG-444. The highest level of total galactosylation reached was 64.6% with 250 uM ABA (FIG. 12B). The maximum fucosylation and galactosylation levels achieved in this experiment were lower than those seen with the Dox-inducible systems. However, the uninduced levels of fucosylation and galactosylation are lower in the ABA-inducible system, likely due to less leaky expression in the absence of ABA than when compared to the Dox-inducible system.

### Example 6: Simultaneous Regulation of FUT8 and β4GALT1 Genes Under Inducible Promoters

For simultaneous and independent control of FUT8 and β4GALT1 genes, pMC11 and pMC4.1 circuits were integrated into the triple landing pad cell line containing both FUT8 and β4GALT1 knockouts. pMC11 was chosen for FUT8 expression because the ABA-inducible system results in a tighter regulation of fucosylation with no inducer. While afucosylation is easily achievable with knockouts, it is important that the lower range of total fucosylation be accessible for broad effector function modulation. pMC4.1 was chosen for β4GALT1 expression because the Dox-inducible system results in higher levels of galactosylation upon induction, which are desirable for effector function studies and are required for subsequent sialylation.

At 1000 nM Dox concentration, the levels of total galactosylation only reached intermediate levels at around 45%. At this level of galactosylation, a range from 1.6% to 74.1% total fucosylation was attained (FIG. 13A). This range can be further assayed for low and high levels of galactosylation. At very low levels of fucosylation (absence of ABA), a range from 4.6% to 59.1% total galactosylation was demonstrated (FIG. 13B). This range can be further assayed for mid and high levels of fucosylation. The result of the dual inducible expression systems is access to the full range of fucosylation and galactosylation never before established for mAbs in vivo.

### Example 7: JUG-444 Effector Function Study

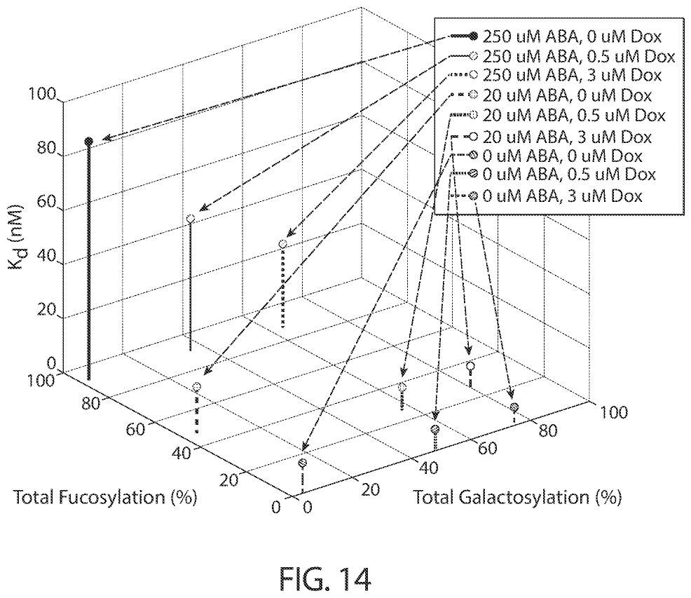

The ADCC is initiated by the binding of Fab portion of IgG to the target antigen on target cells and Fc portion of IgG to FcγRIIIa on the surface of effector cells. The effector cells release cytotoxic factors that cause the death of the antibody-covered target cells. The glycosylation profile of the IgG can impact the binding of IgG to FcγRIIIa. The binding affinity of IgG to FcγRIIIa can be determined by surface plasmon resonance (SPR) analysis. JUG-444 with nine different glycosylation profiles (FIG. 14) were achieved by inducing variable FUT8 and β4GALT1 expression from pMC11 and pMC4.1 circuits in the double knockout cell line. The binding affinity of these nine antibody glycoforms to FcγRIIIa were then analyzed by SPR. The lowest binding affinity (K_{D}=88.7 nM) observed is of JUG-444 with 91.5% fucosylation and 1.9% galactosylation. The highest binding affinity (K_{D}=6.2 nM) observed is JUG-444 with 1.2% fucosylation and 75.4% galactosylation. There is a clear relationship between Fc fucosylation and FcγRIIIa binding, where lower fucosylation levels have increased binding affinity of Fc to FcγRIIIa. In addition, higher Fc galactosylation levels also have increased binding affinity, but not as dramatically as seen with changes in fucosylation. This confirms previously reported effects of afucosylation and hypergalactosylation increasing ADCC (Liu S. D., et al., Cancer Immunol. Res. 2015 February; 3(2): 173-83; Thomann M., et al., Mol. Immunol. 2016 May; 73: 69-75).

### Example 8: ST6GAL1 Expression Under a Constitutive Promoter

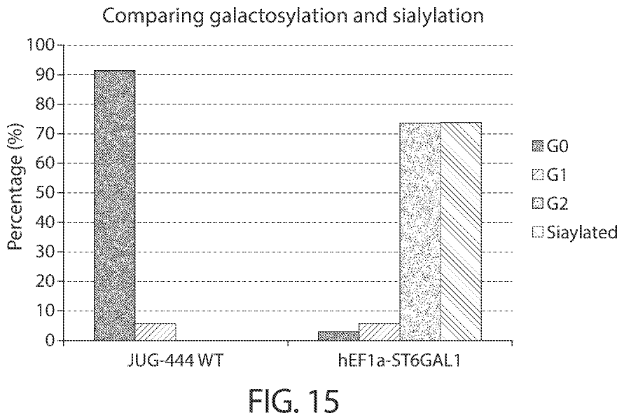

Surprisingly, expression of the pMC2 and pMC20 circuits (see FIG. 4 and FIG. 8) in the β4GALT1 KO cell line dramatically increased total galactosylation and sialylation levels from 6.4% and 0.0% in wild-type JUG-444 to 79.7% and 74.0%, respectively, in the modified JUG-444 (FIG. 15). Interestingly, endogenous FUT8 expression resulted in 81.0% fucosylation compared to 94.6% in WT JUG-444. This interplay between fucosylation, galactosylation, and sialylation was not previously demonstrated and was completely unexpected.

### Example 9: Conclusions

This study demonstrates that synthetic biology approaches can be used to modulate mAb fucosylation and galactosylation independently and in a controlled manner. While there has been some progress in engineering the expression host or enzymatically modifying purified mAb, this is the first case in which endogenous FUT8 and β4GALT1 genes have been knocked out and synthetic versions integrated into the genome under inducible promoters. Therefore, this is also the first instance in which these glycosyltransferase genes have been stably expressed at tunable levels, allowing for a wide range of galactosylated and fucosylated species not easily accessible before in vivo. The power of simultaneous, precise modulation of each gene means mAbs can now be engineered in a cell-line manufacturing process with specific glycosylation patterns suited for particular Fc-mediated effector functions or to produce biopharmaceutical with any desirable level of fucosylation and galactosylation. This approach is not limited to FUT8 and β4GALT1 genes. Now that high levels of galactosylation can be reliably achieved, altering levels of sialylation also can be achieved and should open doors to developing new mAb therapeutics with desired potency, safety/immunogenicity and pharmokinetic properties. Importantly, this method should be broadly applicable beyond mAb therapeutics to any new recombinant protein therapeutics.

### Example 10. Methods and Materials for Examples 11-14

Landing pad cell construction: Multi-landing pad CHO cell lines were constructed targeting LP2 and LP20 loci. Briefly, donor vectors containing hEF1a-attP-BxB1-EBFP-P2A-Bla (cassette1) or hEF1a-attP-BxB1-GA-EYFP-P2A-Hygro (cassette2), with left and right homologous arms were co-transfected with pSpCas9(BB) vector and GeneArt® CRISPR U6 Strings™ DNA using Neon electroporation. After CRISPR/Cas9-mediated homologous recombination, BFP positive cells were single cell sorted by FACS.

Vector construction: Gibson assembly cloning method was used to insert promoters and gene fragments into entry vectors. Expression vectors were constructed by LR cloning with destination plasmids.

CHO cell culture and fed-batch culture: Suspension CHO cells were grown on serum-free CD-CHO medium supplemented with 8 mM L-glutamine. Cultures were incubated in shaking incubator (37° C.) with 7% CO₂ at 130 rpm. Seven-day fed-batch cultures were performed in 250-mL in Erlenmeyer flask containing 50 mL working volume or 125-mL in Erlenmeyer flask containing 25 mL working volume. On day 0, the cells were seeded at a seeding density of 1.5×10⁶ cells/mL. From day 3 through day 6, pH was titrated with 0.94 M Na₂CO₃/0.06 M K₂CO₃ twice a day and cell culture were supplemented with Cell Boost 5 Supplement (Hyclone) and 20% (w/v) D-glucose. On day 7, cultures were harvested and clarified media.

RNA extraction and RT-qPCR: Total RNA was extracted with TRIzol Reagent (Invitrogen) and 1 ug was used for cDNA synthesis using QuantiTect Reverse Transcription kit (Qiagen). mRNA expressions were quantified by SYBR Green RT-qPCR assay in LightCycler® 96 system (Roche). Relative gene expressions were analyzed by the ΔΔC_{T} method using B-actin as the reference gene for normalization.

### Example 11. Constitutive FUT8 Expressions Using Promoter Mini Libraries Results in Mostly High Fucosylation Level of mAbs

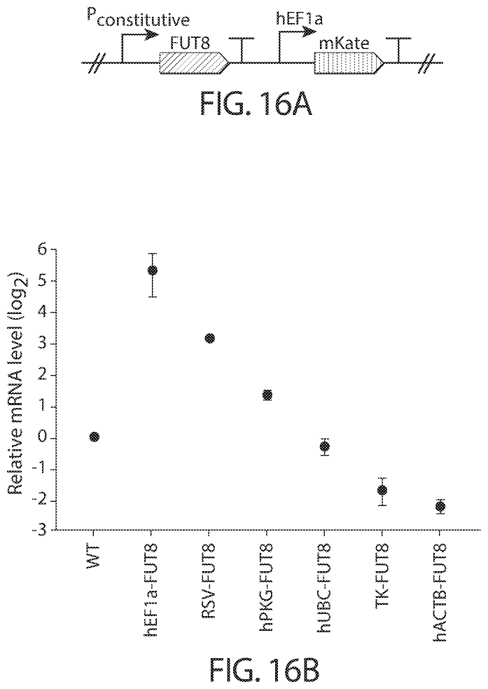

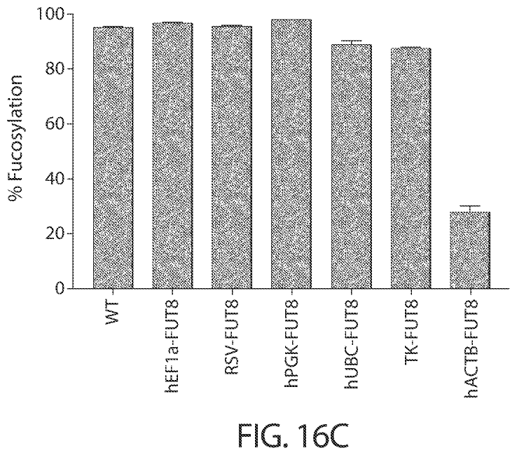

To restore the FUT8 expression in knockout cell lines, genetic circuits expressing synthetic version of FUT8 under commonly used mammalian constitutive promoters were introduced (hEF1a, RSV, hPGK, hUBC, HSV-TK, hACTB) (FIG. 16A). Although the FUT8 transcriptional levels of the cells varied (FIG. 16B), FUT8 expression from these circuits resulted in mostly highly fucosylated mAbs (FIG. 16C). First, FUT8 constitutively expressing circuits were integrated into LP20 loci of the FUT8 KO cell line. Genomic integration was confirmed by the florescent marker, mKate expression. After 7 days of the fed-batch cultivation, mAb production was analyzed by HILIC. Cell lines expressing FUT8 under strong constitutive promoters, including hEF1a, RSV, hPGK promoters, all produced more than 91% fucosylated mAbs, corresponding to the endogenous fucosylation level under the same fed-batch condition. Relatively weaker promoters, hUBC, TK, and hACTB, generated 80%, 75%, 30% fucosylated species, respectively. FUT8 mRNA expression of the cell lines was analyzed at day 0 of fed-batch culture. hEF1a-FUT8 cell line expressed nearly 40-fold increase relative to wild-type; however, mAb fucosylation levels were similar to WT. In comparison between the TK-FUT8 cell line and the hACTB-FUT8 cell line, mRNA level differed by less than 2-fold, but fucosylation levels differed significantly (70% vs 30%). All cell lines produced higher galactosylated species than WT level (10.9%), mostly represented as G1 species.

TABLE 3
Constitutive promoters used in this study.

| Promoter name | Promoter size | Origin |
|---|---|---|
| hEF1a (human Elongation | 1174 bp | human |
| Factor 1 alpha) | | |
| RSV (Rous sarcoma virus) | 228 bp | Retrovirus Viral |
| | | promoter |
| hPGK (human phosphoglycerate | 541 bp | human |
| kinase 1 promoter) | | |
| hUBC (human Ubiquitin C promoter) | 403 bp + 1^{st} | human |
| | intron 814 bp | |
| TK (Herpes simplex virus (HSV) | 252 bp | Retrovirus herpes |
| thymidine kinase promoter) | | simplex virus |
| hACTB (human Actin Beta) | 614 bp | human |

TABLE 4
Glycosylation analysis of mAb produced from constitutive expressing cell lines.

| | | Total | | | G2 | G1 | G0 |
|---|---|---|---|---|---|---|---|
| Cell | | Galactosylation | Fucosylation | Sialylation | Species | Species | Species |
| line | Vector design | (%) | (%) | (%) | (%) | (%) | (%) |
| Jug444 WT | | 10.88 ± 0.48 | 95.37 ± 0.34 | 0.61 ± 0.05 | 1.3 ± 0.05 | 9.58 ± 0.5 | 87.83 ± 0.35 |
| MC1 | hEF1a-FUT8_hEF1a-mKate | 18.71 ± 0.61 | 96.99 ± 0.07 | 0.28 ± 0.03 | 1.68 ± 0.34 | 17.03 ± 0.29 | 78.75 ± 0.57 |
| GJ118 | RSV-FUT8_hEF1a-mKate | 20.5 ± 0.17 | 95.89 ± 0.18 | 1.73 ± 0.13 | 3.45 ± 0.05 | 17.04 ± 0.21 | 77.19 ± 0.18 |
| GJ116 | hUBC-FUT8_hEF1a-mKate | 18.43 ± 1.25 | 89.01 ± 1.17 | 3.4 ± 0.94 | 5.26 ± 1.5 | 13.17 ± 0.29 | 76.91 ± 2.29 |
| GJ117 | TK-FUT8_hEF1a-mKate | 21.86 ± 0.6 | 87.6 ± 0.42 | 1.5 ± 0.26 | 2.93 ± 0.53 | 18.93 ± 0.43 | 76.92 ± 0.53 |
| GJ120 | hACTB-FUT8_hEF1a-mKate | 14.34 ± 2.4 | 27.91 ± 1.79 | 0.45 ± 0 | 1.48 ± 0.67 | 12.86 ± 1.74 | 84 ± 2.95 |
| GJ121 | hPGK-FUT8_hEF1a-mKate | 22.46 ± 0.37 | 98.06 ± 0.02 | 0.93 ± 0.03 | 2.44 ± 0.03 | 20.01 ± 0.33 | 76.02 ± 0.38 |

### Example 12. Reduced Fucosylation in mAbs Resulted from Intronic miRNA Circuits

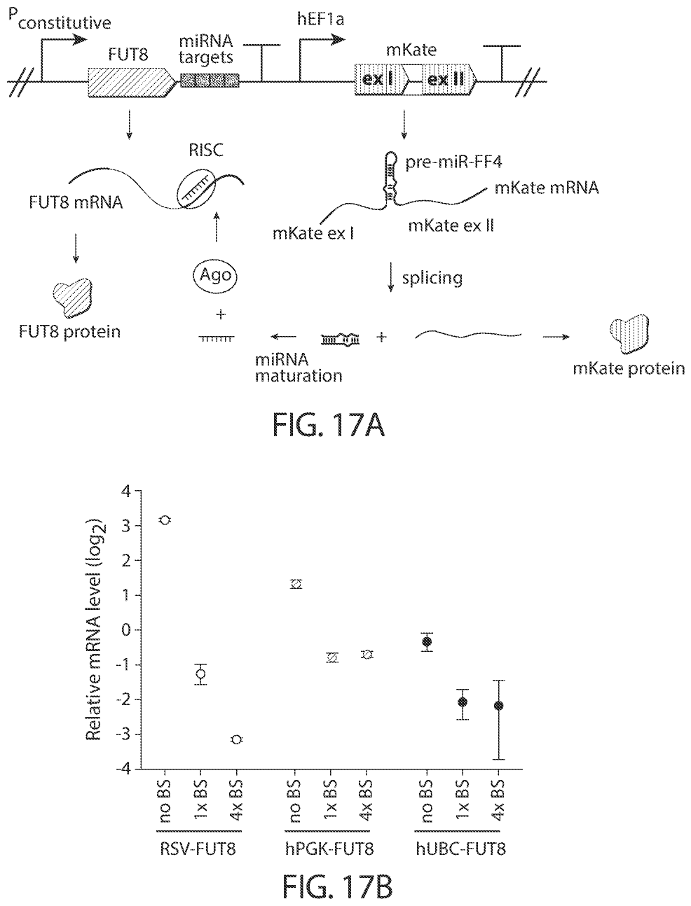

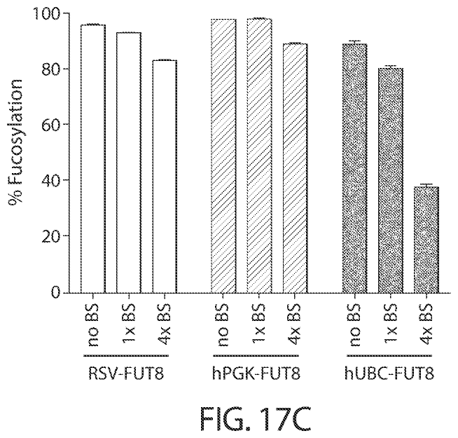

To reduce the fucosylation level of mAb, miRNA binding sites were inserted in the 3′ UTR of synthetic FUT8 sequences (FIG. 17A). The binding sites perfectly complemented the sequence of the miRNA allowing the formation of RNA-induced silencing complex with the transcribed mRNA. The synthetic miRNA miR-FF4 was used, which does not target any endogenous CHO genome. A sequence encoding the miR-FF4 miRNA was embedded in the intronic region of the mKate florescent protein. In this way, during the pre-mRNA splicing process, intronic miRNA are matured. The matured miRNA can then bind the mRNA and induce translational repression and destabilization. Three promoters exhibiting intermediate strengths (RSV, hPGK and hUBC) were selected to maximize the range of repression. To increase the repression level, cells bearing four repeats of miRNA binding sites in the 3′ UTR were created. Relative FUT8 mRNA expression was normalized to the wild-type cell line. All miRNA genetic circuits cells showed reduced mRNA expression compared to parental genetic circuits cell lines (FIG. 17B). While cells having circuits with RSV-FUT8 and 4× binding sites showed approximately 16-fold reduced mRNA level relative to the 1× binding site counterpart cells, cells having circuits with hPGK-FUT8 and hUBC-FUT8 with 4× binding sites showed no significant difference in mRNA level relative to their respective 1× binding site cells. However, reduced fucosylation levels of mAb were found in all cell lines having circuits with 4× binding sites relative to their respective 1× binding site cell lines (FIG. 17C). RSV or hPGK 1× binding site circuits showed minimal differences (2.6% and 0.2%) in fucosylation levels relative to circuit without binding sites (FIG. 17C). Overall, relative FUT8 mRNA expressions were reduced compared with the cells without binding sites. In additions, there was no significant difference in relative glycoform abundance except the fucosylation.

TABLE 5
Glycan analysis from mAbs produced from intronic miRNA circuit cell lines.

| | | | Total | | G2 | G1 | G0 |
|---|---|---|---|---|---|---|---|
| Cell | | Fucosylation | Galactosylation | Sialylation | Species | Species | Species |
| line | Vector design | (%) | (%) | (%) | (%) | (%) | (%) |
| GJ127 | hPGK-FUT8-1xFF4- | 97.86 ± 0.14 | 24.02 ± 0.67 | 1.11 ± 0.07 | 3.12 ± 0.13 | 20.9 ± 0.55 | 75.07 ± 0.76 |
| | bs_hef1a-mKate-intr-FF4 | | | | | | |
| GJ128 | hPGK-FUT8-4xFF4- | 89.43 ± 0.22 | 23.83 ± 0.67 | 0.92 ± 0.16 | 3.12 ± 0.66 | 20.71 ± 0.16 | 75.26 ± 0.74 |
| | bs_hef1a-mKate-intr-FF4 | | | | | | |
| GJ131 | hUBC-FUT8-1xFF4- | 80.06 ± 0.96 | 23.94 ± 1.93 | 1.21 ± 0.79 | 3.67 ± 1.41 | 20.27 ± 0.53 | 75.14 ± 2.26 |
| | bs_hef1a-mKate-intr-FF4 | | | | | | |
| GJ132 | hUBC-FUT8-4xFF4- | 37.55 ± 1.11 | 18.26 ± 2.09 | 1.15 ± 0.73 | 3.79 ± 1.69 | 14.47 ± 0.43 | 79.33 ± 3.05 |
| | bs_hef1a-mKate-intr-FF4 | | | | | | |
| GJ133 | RSV-FUT8-1xFF4- | 93.24 ± 0.13 | 24.55 ± 0.43 | 1.16 ± 0.07 | 3.37 ± 0.26 | 21.18 ± 0.24 | 74.51 ± 0.44 |
| | bs_hef1a-mKate-intr-FF4 | | | | | | |
| GJ134 | RSV-FUT8-4xFF4- | 83.25 ± 0.3 | 22.09 ± 0.55 | 0.56 ± 0.27 | 3.16 ± 0.31 | 18.92 ± 0.62 | 76.05 ± 0.59 |
| | bs_hef1a-mKate-intr-FF4 | | | | | | |

### Example 13. Highly Reduced Fucosylation in mAbs Achieved from U6 Promoter-Transcribed miRNAs Circuits

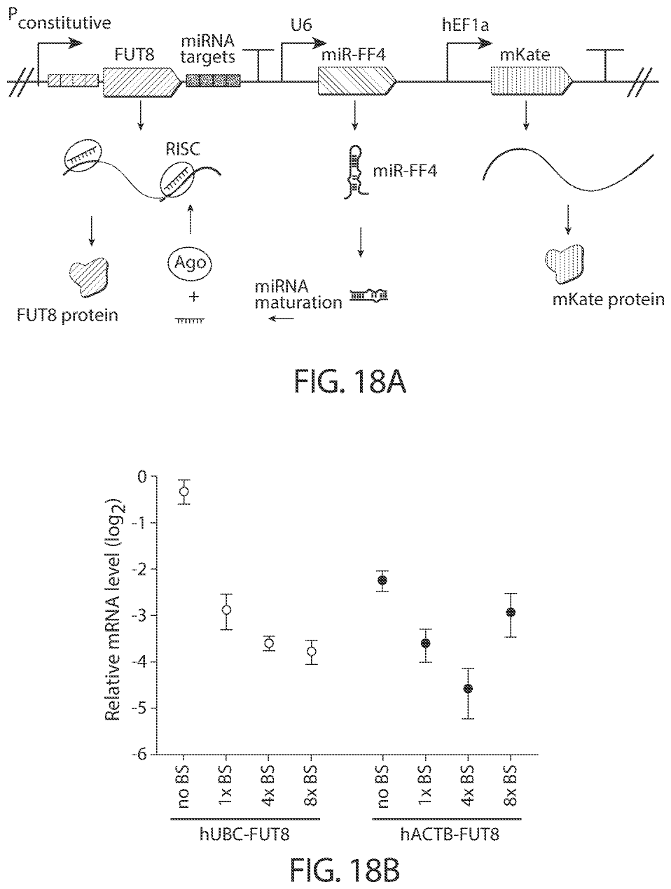

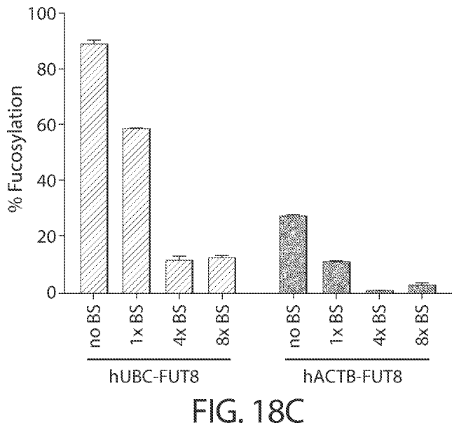

To further reduce fucosylation levels, additional miRNA expressing circuits were constructed (FIG. 18A). Here, miR-FF4 was produced from the U6 promoter to express miRNAs independent from mKate expression. Additionally, miRNA binding sites (4×) were added to the 5′ UTR of the FUT8 cDNA sequence. Thus, circuits included 1) one miR-FF4 binding site in the 3′ UTR, 2) 4 miR-FF4 binding sites in the 3′ UTR, and 3) 4 miR-FF4 binding sites in the 3′ UTR and 4 miR-FF4 binding sites in the 5′ UTR. Two constitutive promoters were used (hUBC and hACTB) which produced mAb with 89% and 28% of fucosylation level without any miRNA regulations. It was found that 1×miR-FF4 binding site in the 3′ UTR reduced fucosylation levels (FIG. 18C). GJ135, hUBC promoter driven FUT8 circuit with miR-FF4 1× binding site, showed a 59.1% fucosylation level, which is 21% more reduced fucosylation level than GJ131 (intronic FF4 1× binding circuit). Additional 3× binding sites in the 3′ UTR (GJ136) resulted in 12.0% fucosylated mAbs, which is 6.4-fold decrease from the no-binding site circuits. However, interestingly, no significant difference was seen in fucosylation levels between GJ136 and GJ137, which has 4× additional miR-FF4 binding sites in the 5′ UTR. Likewise, GJ139, hUBC promoter driven FUT8 circuit with miR-FF4 1× binding site generated antibodies with 14% fucosylated mAbs. Similarly, GJ138, hACTB promoter driven FUT8 circuit with miR-FF4 1× binding site, produced 14% fucosylated mAbs, which is 2 fold decrease from circuit without miRNA binding sites. GJ139 which has 4×miR-FF4 binding sites showed highly repressed levels of fucosylation, 0.9%. We found that cell lines with additional 4×miR-FF4 binding sites in the 5′ UTR, GJ140, generated slightly higher levels of fucosylation, 3.2%.

TABLE 6
Glycan analysis from mAbs produced from U6-transcribed miRNA circuit cell lines.

| | | | Total | | G2 | G1 | G0 |
|---|---|---|---|---|---|---|---|
| Cell | | Fucosylation | Galactosylation | Sialylation | Species | Species | Species |
| line | Vector design | (%) | (%) | (%) | (%) | (%) | (%) |
| GJ135 | hUBC-FUT8-1xFF4- | 59.09 ± 0.3 | 20.28 ± 0.25 | 0.65 ± 0.12 | 2.05 ± 0.18 | 18.23 ± 0.09 | 79.24 ± 0.21 |
| | bs_U6-FF4_hef1a-mKate | | | | | | |
| GJ136 | hUBC-FUT8-4xFF4- | 11.95 ± 1.17 | 15.35 ± 2.1 | 0.36 ± 0.2 | 1.61 ± 0.62 | 13.74 ± 1.5 | 82.92 ± 2.8 |
| | bs_U6-FF4_hef1a-mKate | | | | | | |
| GJ137 | hUBC-4xFF4-bs-FUT8-4xFF4- | 12.96 ± 0.68 | 16.82 ± 0.15 | 0.52 ± 0.08 | 1.72 ± 0.15 | 15.1 ± 0.05 | 81.47 ± 0.18 |
| | bs_U6-FF4_hef1a-mKate | | | | | | |
| GJ138 | hACTB-FUT8-1xFF4- | 14.06 ± 0.48 | 12.46 ± 0.55 | 0.11 ± 0.04 | 1.04 ± 0.2 | 11.42 ± 0.37 | 86.17 ± 0.47 |
| | bs_U6-FF4_hef1a-mKate | | | | | | |
| GJ139 | hACTB-FUT8-4xFF4- | 0.91 ± 0.01 | 11.97 ± 0.13 | 0.4 ± 0.07 | 1.36 ± 0.1 | 10.6 ± 0.1 | 86.07 ± 0.16 |
| | bs_U6-FF4_hef1a-mKate | | | | | | |
| GJ140 | hACTB-4xFF4-bs-FUT8-4xFF4- | 3.23 ± 0.79 | 16.05 ± 0.99 | 1.71 ± 0.55 | 3 ± 0.53 | 13.05 ± 0.57 | 78.97 ± 1.67 |
| | bs_U6-FF4_hef1a-mKate | | | | | | |

### Example 14. Engineered Cells Maintained the Cell Line Stability During the Long-Term Culture

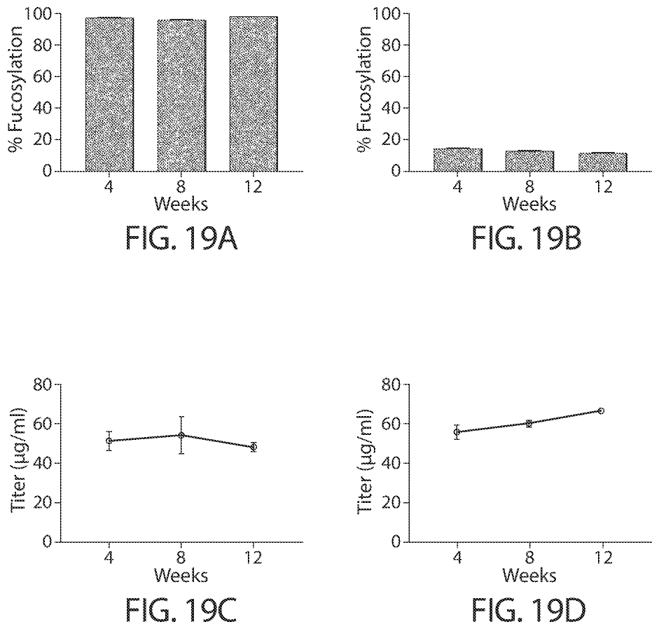

It is critical to maintain the quality of protein and titer for therapeutic recombinant protein production using CHO cells. To evaluate engineered cell line stability, MC1 and GJ138 cell pools were sub-cultured for three months and fed-batch culture was performed every four weeks. Antibodies from harvested clarified media were analyzed by HILIC and measured the titer using Octet platform. There was no significant decrease in titer during 3-month culture, approximately 90 generations (FIGS. 19A-19D). The MC1 pools produced highly fucosylated antibodies (96.98%) at all three time points (4, 8, and 12 weeks), and other glycosylation profiles such as galactosylation (18.45%) and sialyation levels (0.44%) were maintained. The relative proportions of G0, G1 and G2 glycans also were constant, with 78.93%, 16.88%, and 1.41%, respectively. GJ138, which has miRNA 1× binding site at 3′ UTR of synthetic FUT8 sequence, showed low fucosylation levels (12.83%) throughout the 90 generations. Total galactosylation levels (13.76%) were approximately 4.7% less than MC1 pool, however, they maintained galactosylation levels within the cell pool. Sialyation in the GJ138 cell pool showed reduced levels (0.27%) relative to MC1. The relative abundance of G0, G1, and G2 species of GJ138 retained for long term culture. The protein productivities in these cell pool showed no significant change over 90 generations.

TABLE 7
Glycan analysis of mAb for cell line stability.

| | | Total | | G0 | G1 | G2 |
|---|---|---|---|---|---|---|
| | Fucosylated (%) | Galactosylated (%) | Sialylated (%) | Species | Species | Species |
| MC1-P30 | 97.19 ± 0.09 | 16.96 ± 1.67 | 0.41 ± 0.13 | 80.37 ± 1.59 | 15.58 ± 1.15 | 1.37 ± 0.52 |
| MC1-P60 | 95.69 ± 0.51 | 19.44 ± 0.59 | 0.54 ± 0.04 | 76.46 ± 0.41 | 17.82 ± 0.59 | 1.62 ± 0.10 |
| MC1-P90 | 98.05 ± 0.03 | 18.45 ± 1.78 | 0.38 ± 0.21 | 79.94 ± 1.73 | 17.23 ± 1.34 | 1.22 ± 0.45 |
| average | 96.98 ± 1.09 | 18.28 ± 1.66 | 0.44 ± 0.13 | 78.93 ± 2.08 | 16.88 ± 1.39 | 1.41 ± 0.31 |
| GJ138-P30 | 14.06 ± 0.48 | 12.46 ± 0.55 | 0.11 ± 0.04 | 86.17 ± 0.47 | 11.42 ± 0.37 | 1.04 ± 0.20 |
| GJ138-P60 | 12.74 ± 0.48 | 13.52 ± 0.87 | 0.16 ± 0.16 | 85.17 ± 1.05 | 12.45 ± 0.61 | 1.07 ± 0.32 |
| GJ138-P90 | 11.68 ± 0.32 | 15.29 ± 0.49 | 0.46 ± 0.14 | 83.23 ± 0.57 | 13.57 ± 0.28 | 1.72 ± 0.22 |
| average | 12.83 ± 0.89 | 13.76 ± 1.42 | 0.27 ± 0.21 | 84.85 ± 1.50 | 12.48 ± 1.03 | 1.27 ± 0.43 |

TABLE 8
mAb titer from long-term fed-batch culture.

| | Titer (μg/ml) |
|---|---|
| MC1-P30 | 51.67 ± 4.82 |
| MC1-P60 | 54.67 ± 9.35 |
| MC1-P90 | 48.63 ± 2.24 |
| GJ138-P30 | 55.87 ± 2.33 |
| GJ138-P60 | 61.20 ± 1.22 |
| GJ138-P90 | 64.97 ± 0.21 |

### REFERENCES

1. Bartel D. P., MicroRNAs: Target Recognition and Regulatory Functions, Cell. 2009 Jan. 23; 136(2): 215-33.
2. Chiu M. L. and Gilliland G. L., Engineering antibody therapeutics, Curr. Opin. Struct. Biol. 2016 June; 38: 163-73.
3. Dow L. E., Nasr Z., Saborowski M., Ebbesen S. H., Manchado E., Tasdemir N., Lee T., Pelletier J., and Lowe S. W., Conditional Reverse Tet-Transactivator Mouse Strains for the Efficient Induction of TRE-Regulated Transgenes in Mice, PLoS One. 2014 Apr. 17; 9(4): e95236.
4. Duportet X., Wroblewska L., Guye P., Li Y., Eyquem J., Rieders J., Rimachala T., Batt G., and Weiss R., A platform for rapid prototyping of synthetic gene networks in mammalian cells, Nucleic Acids Res. 2014 Dec. 1; 42(21): 13440-51.
5. Ferreira J. P., Overton K. W., and Wang C. L., Tuning gene expression with synthetic upstream open reading frames, Proc. Natl. Acad. Sci. U.S.A. 2013 Jun. 9; 110(28): 11284-89.
6. Gaidukov L., Wroblewska L., Teague B., Nelson T., Zhang X., Liu Y., Jagtap K., Mamo S., Tseng W. A., Lowe A., Das J., Bandara K., Baijuraj S., Sumers N. M., Lu T. K., Zhang L., Weiss R., Multi-landing pad DNA integration platform for mammalian cell engineering, Nucleic Acids Res. 2018 May 4; 46(8): 4072-86.
7. Gao Y., Xiong X., Wong S., Charles E. J., Lim W. A., and Qi L. S., Complex transcriptional modulation with orthogonal and inducible dCas9 regulators. Nat. Methods. 2016 December; 13(12): 1043-49.
8. Ho S. C., Koh E. Y., van Beers M., Mueller M., Wan C., Teo G., Song Z., Tong Y. W., Bardor M., and Yang Y., Control of IgG LC:HC ratio in stably transfected CHO cells and study of the impact on expression, aggregation, glycosylation and conformational stability, J. Biotechnol. 2013 Jun. 10; 165(3-4): 157-66.
9. Inniss M. C., Bandara K., Jusiak B., Lu T. K., Weiss R., Wroblewska L., and Zhang L., A novel Bxbl integrase RMCE system for high fidelity site-specific integration of mAb expression cassette in CHO Cells, Biotechnol. Bioeng. 2017 August; 114(8): 1837-46.
10. Jefferis R., Recombinant antibody therapeutics: the impact of glycosylation on mechanisms of action, Trends Pharmacol. Sci. 2009 July; 30(7): 356-62.
11. Kanda Y., Imai-Nishiya H., Kuni-Kamochi R., Mori K., Inoue M., Kitajima-Miyama K., Okezaki A., lida S., Shitara K., and Satoh M., Establishment of a GDP-mannose 4,6-dehydratase (GMD) knockout host cell line: A new strategy for generating completely non-fucosylated recombinant therapeutics, J. Biotechnol. 2007 Jun. 20; 130(3): 300-10.
12. Kaneko Y., Nimmerjahn F., and Ravetch J. V., Anti-inflammatory activity of immunoglobulin G resulting from Fc sialylation, Science. 2006 Aug. 4; 313(5787): 670-73.
13. Li F., Vijayasankaran N., Shen A. Y., Kiss R., and Amanullah A., Cell culture processes for monoclonal antibody production, MAbs. 2010 September-October; 2(5): 466-79.
14. Liang F. S., Ho W. Q., and Crabtree G. R., Engineering the ABA plant stress pathway for regulation of induced proximity, Sci. Signal. 2011 Mar. 15; 4(164): rs2.
15. Liu L., Antibody glycosylation and its impact on the pharmacokinetics and pharmacodynamics of monoclonal antibodies and Fc-fusion proteins, J. Pharm. Sci. 2015 June; 104(6): 1866-84.
16. Liu S. D., Chalouni C., Young J. C., Junttila T. T., Sliwkowski M. X., and Lowe J. B., Afucosylated antibodies increase activation of FcγRIIIa-dependent signaling components to intensify processes promoting ADCC, Cancer Immunol. Res. 2015 February; 3(2): 173-83.
17. Mori K., Kuni-Kamochi R., Yamane-Ohnuki N., Wakitani M., Yamano K., Imai H., Kanda Y., Niwa R., lida S., Uchida K., Shitara K., and Satoh M., Engineering Chinese hamster ovary cells to maximize effector function of produced antibodies using FUT8 siRNA, Biotechnol. Bioeng. 2004 Dec. 30; 88(7): 901-8.
18. Mullick A., Xu Y., Warren R., Koutroumanis M., Builbault C., Broussau S., Malenfant F., Bourget L., Lamoureux L., Lo R., Caron A. W., Pilotte A., and Massie B., The cumate gene-switch: A system for regulated expression in mammalian cells, BMC Biotechnol. 2006 Nov. 3; 6: 43.
19. Nissim L., Wu M. R., Pery E., Binder-Nissim A., Suzuki H. I., Stupp D., Wehrspaun C., Tabach Y., Sharp P. A., and Lu T. K., Synthetic RNA-Based Immunomodulatory Gene Circuits for Cancer Immunotherapy, Cell. 2017 Nov. 16; 171(5): 1138-50.
20. Shang T. Q., Saait A., Toler K. N., Mo J., Li H., Matlosz T., Lin X., Schenk J., Ng C. K., Duffy T., Porter T. J., and Rouse J. C., Development and application of a robust N-glycan profiling method for heightened characterization of monoclonal antibodies and related glycoproteins, J. Pharm. Sci. 2014 July; 103(7): 1967-78.
21. Sun T., Li C., Han L., Jiang H., Xie Y., Zhang B., Qian X., Lu H., and Zhu J., Functional knockout of FUT8 in Chinese hamster ovary cells using CRISPR/Cas9 to produce a defucosylated antibody, Eng. Life Sci. 2015 Jul. 21; 15(6): 660-66.
22. Thomann M., Reckermann K., Reusch D., Prasser J., and Tejada M. L., Fc-galactosylation modulates antibody-dependent cellular cytotoxicity of therapeutic antibodies, Mol. Immunol. 2016 May; 73: 69-75.
23. Vaquerizas J. M., Kummerfeld S. K., Teichmann S. A., and Luscombe N. M., A census of human transcription factors: function, expression and evolution, Nat. Rev. Genet. 2009 April; 10(4): 252-63.
24. Weiner L. M., Murray J. C., and Shuptrine C. W., Antibody-based immunotherapy of cancer, Cell. 2012 Mar. 16; 148(6): 1081-84.
25. Wingender E., Schoeps T., and Donitz, J., TFClass: An expandable hierarchical classification of human transcription factors, Nucleic Acids Res. 2013 January; 41.
26. Yamane-Ohnuki N., Kinoshita S., Inoue-Urakubo M., Kusunoki M., Lida S., Nakano R., Wqakitani M., Niwa R., Sakurada M., Uchida K., Shitara K., and Satoh M., Establishment of FUT8 knockout Chinese hamster ovary cells: An ideal host cell line for producing completely defucosylated antibodies with enhanced antibody-dependent cellular cytotoxicity, Biotechnol. Bioeng. 2004 Sep. 5; 87(5): 614-22.
27. Yang Z., Wang S., Halim A., Schulz M. A., Frodin M., Rahman S. H., Vester-Christensen M. B., Behrens C., Kristensen C., Vakhurshev S. Y., Bennett E. P., Wandall H. H., and Clausen H., Engineered CHO cells for production of diverse, homogeneous glycoproteins, Nat. Biotechnol. 2015 August; 33(8): 842-44.
28. Zong H., Han L., Ding K., Wang J., Sun T., Zhang X., Cagliero C., Jian H., Xie Y., Xu J., Zhang B., and Zhu J., Producing defucosylated antibodies with enhanced in vitro antibody-dependent cellular cytotoxicity via FUT8 knockout CHO-S cells. Eng. Life Sci. 2017 Feb. 23; 17(7): 801-8.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An engineered, non-naturally occurring eukaryotic cell comprising a modified genome, wherein the modified genome comprises:
(a) a knockout of at least one endogenous polynucleic acid sequence encoding a glycan modifying enzyme; and
(b) an integration of at least one polynucleic acid sequence comprising a sequence encoding a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme.

2. The engineered, non-naturally occurring eukaryotic cell of claim 1, wherein the tunable control element in (b) is selected from the group consisting of an inducible promoter element, a synthetic promoter panel, a miRNA response element, and an ORF control element.

3. The engineered, non-naturally occurring eukaryotic cell of claim 1, wherein the engineered, non-naturally occurring eukaryotic cell comprises: (b) an integration of at least two polynucleic acid sequences, wherein each polynucleic acid sequence comprises the sequence of a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme.

4. The engineered, non-naturally occurring eukaryotic cell of claim 3, wherein the tunable control element of each of the at least two polynucleic acid sequences in (b) is unique.

5. The engineered, non-naturally occurring eukaryotic cell of claim 3 or claim 4, wherein the tunable control element of at least one of the at least two polynucleic acid sequences in (b) is selected from the group consisting of an inducible promoter element, a synthetic promoter panel, a miRNA response element, and an ORF control element.

6. The engineered, non-naturally occurring eukaryotic cell of claim 2-5, wherein the inducible promotor is a chemically-regulated promoter or a physically-regulated promoter.

7. The engineered, non-naturally occurring eukaryotic cell of claim 6, wherein the chemically-regulated promoter comprises a TRE-Tight promoter sequence or a PhlF-activatable promoter sequence.

8. The engineered, non-naturally occurring eukaryotic cell of any one of claims 1-7, wherein the glycan modifying enzyme in (a) is selected from the group consisting of a fucosyltransferase, a galactosyltransferase, a sialyltransferase, an oligosaccharyltransferase, a glycosidase, a mannosidase, and a monoacylglycerol acetyltransferase.

9. The engineered, non-naturally occurring eukaryotic cell of any one of claims 1-8, wherein the glycan modifying enzyme in (a) is FUT8.

10. The engineered, non-naturally occurring eukaryotic cell of any one of claims 1-8, wherein the glycan modifying enzyme in (a) is β4GALT1.

11. The engineered, non-naturally occurring eukaryotic cell of any one of claims 1-8, wherein the genome of the engineered, non-naturally occurring eukaryotic cell comprises a knockout of at least two glycan modifying enzymes, wherein one of the at least two glycan modifying enzymes is FUT8 and one of the at least two glycan modifying enzymes is β4GALT1.

12. The engineered, non-naturally occurring eukaryotic cell of any one of claims 1-11, wherein the engineered, non-naturally occurring eukaryotic cell further comprises an integration of a polynucleic acid sequence comprising the sequence of a protein of interest operably linked to a constitutive or inducible promoter, wherein the protein of interest can be modified by the addition of a glycan.

13. The engineered, non-naturally occurring eukaryotic cell of claim 12, wherein the protein of interest is an immunoglobulin.

14. The engineered, non-naturally occurring eukaryotic cell of claim 13, wherein the immunoglobulin belongs to the IgA, IgD, IgE, IgG, or IgM class.

15. The engineered, non-naturally occurring eukaryotic cell of claim 13 or claim 14, wherein the immunoglobulin is an IgG1, IgG2, IgG3, or IgG4 immunoglobulin.

16. The engineered, non-naturally occurring eukaryotic cell of any one of claims 1-15, wherein the eukaryotic cell is a CHO cell, a COS cell, a NS0 cell, Sp2/0 cell, BHK cell, HEK293 cell, HEK293-EBNA1 cell, HEK293-F cell, HT-1080 cell, HKB-11, CAP cell, HuH-7 cell, or a PER.C6 cell.

17. The engineered, non-naturally occurring eukaryotic cell of any one of claims 1-16, wherein the eukaryotic cell is a CHO cell.

18. The engineered, non-naturally occurring eukaryotic cell of any one of claims 1-17, wherein the integration of the at least one polynucleic acid sequence comprising the sequence of a functional copy of a glycan modifying enzyme and/or the integration of the at least one polynucleic acid sequence comprising the sequence of protein of interest operably linked to a constitutive or inducible promoter is at one or more landing pads.

19. A method of generating a glycoprotein comprising a distinct glycan structure, said method comprising expressing at least one protein of interest and at least one glycan modifying enzyme in an engineered, non-naturally occurring eukaryotic cell comprising a modified genome, wherein the modified genome comprises:
(a) a knockout of at least one endogenous polynucleic acid sequence encoding a glycan modifying enzyme;
(b) an integration of at least one polynucleic acid sequence comprising a sequence encoding a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme; and
(c) an integration of at least one polynucleic acid sequence comprising a sequence encoding a protein of interest operably linked to a constitutive or inducible promoter, wherein each of the at least one protein of interest can, when expressed as a protein, be modified by the addition of a glycan.

20. The method of claim 19, wherein the tunable control element in (b) is selected from the group consisting of an inducible promoter element, a synthetic promoter panel, a miRNA response element, and an ORF control element.

21. The method of claim 20, wherein the engineered, non-naturally occurring eukaryotic cell comprises: (b) an integration of at least two polynucleic acid sequences, wherein each polynucleic acid sequence comprises the sequence of a functional copy of a glycan modifying enzyme knocked out in (a), wherein the sequence encoding the functional copy of a glycan modifying enzyme is operably linked to a tunable control element that controls mRNA and/or protein expression of the glycan modifying enzyme.

22. The method of claim 21, wherein the tunable control element of each of the at least two polynucleic acid sequences in (b) is unique.

23. The method of claim 21 or claim 22, wherein the tunable control element of at least one of the at least two polynucleic acid in (b) is selected from the group consisting of an inducible promoter element, a synthetic promoter panel, a miRNA response element, and an ORF control element.

24. The method of claim 20-23, wherein the inducible promotor is a chemically-regulated promoter or a physically-regulated promoter.

25. The method of claim 24, wherein the chemically-regulated promoter comprises a TRE-Tight promoter sequence or a PhlF-activatable promoter sequence.

26. The method of any one of claims 19-25, wherein at least one of the glycan modifying enzymes in (a) is selected from the group consisting of a fucosyltransferase, a galactosyltransferase, a sialyltransferase, an oligosaccharyltransferase, a glycosidase, a mannosidase, and a monoacylglycerol acetyltransferase.

27. The method of any one of claims 19-26, wherein the glycan modifying enzyme in (a) is FUT8.

28. The method of any one of claims 19-26, wherein the glycan modifying enzyme in (a) is β4GALT1.

29. The method of any one of claims 19-26, wherein the genome of the engineered, non-naturally occurring eukaryotic cell comprises a knockout of at least two glycan modifying enzymes, wherein one of the at least two glycan modifying enzymes is FUT8 and one of the at least two glycan modifying enzymes is β4GALT1.

30. The method of claim 29, wherein the protein of interest of (c) is an immunoglobulin.

31. The method of claim 30, wherein the immunoglobulin belongs to the IgA, IgD, IgE, IgG, or IgM class.

32. The method of claim 30 or claim 31, wherein the immunoglobulin is an IgG1, IgG2, IgG3, or IgG4 immunoglobulin.

33. The engineered, non-naturally occurring eukaryotic cell of any one of claims 19-32, wherein the eukaryotic cell is a CHO cell, a COS cell, a NS0 cell, Sp2/0 cell, BHK cell, HEK293 cell, HEK293-EBNA1 cell, HEK293-F cell, HT-1080 cell, HKB-11, CAP cell, HuH-7 cell, or a PER.C6 cell.

34. The method of any one of claims 19-32, wherein the eukaryotic cell is a CHO cell.

35. The method of any one of claims 19-34, wherein the integration of the at least one polynucleic acid sequence comprising the sequence of a functional copy of a glycan modifying enzyme and/or the integration of the at least one polynucleic acid sequence comprising the sequence of protein of interest operably linked to a constitutive or inducible promoter is at one or more landing pads.

36. An immunoglobulin generated by the method of any one of claims 19-35.

37. The immunoglobulin of claim 36, comprising at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% fucosylation.

38. The immunoglobulin of claim 36, comprising at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% galactosylation.

39. The immunoglobulin of claim 36, comprising at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% sialylation.

40. The immunoglobulin of claim 36, comprising:
(a) at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% fucosylation; and/or
(b) at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% galactosylation; and/or
(c) at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, or at least 75% sialylation.

41. A composition comprising at least one immunoglobulin as claimed in any one of claims 36-40.


SEQUENCE LISTING (16 sequences)

SEQ ID NO: 1 - DNA, 20 bp, Artificial Sequence
  Synthetic polynucleotide
ttatttgcttgacatacaca

SEQ ID NO: 2 - DNA, 20 bp, Artificial Sequence
  Synthetic polynucleotide
gtaatcctagtgctatagtg

SEQ ID NO: 3 - DNA, 20 bp, Artificial Sequence
  Synthetic polynucleotide
attgcaacagaaatgtgccg

SEQ ID NO: 4 - DNA, 20 bp, Artificial Sequence
  Synthetic polynucleotide
tagtgagtcagaccaagacg

SEQ ID NO: 5 - DNA, 29 bp, Artificial Sequence
  Synthetic polynucleotide
gaaagatggattgacagggagaggttaag

SEQ ID NO: 6 - DNA, 29 bp, Artificial Sequence
  Synthetic polynucleotide
caggtgatgggagggttttgatgattttc

SEQ ID NO: 7 - DNA, 29 bp, Artificial Sequence
  Synthetic polynucleotide
caggtgatgggagggttttgatgattttc

SEQ ID NO: 8 - DNA, 31 bp, Artificial Sequence
  Synthetic polynucleotide
gagaaccatcacataaactaaggaaaacacc

SEQ ID NO: 9 - DNA, 30 bp, Artificial Sequence
  Synthetic polynucleotide
cttccctttgactccacttctatgaaattg

SEQ ID NO: 10 - DNA, 29 bp, Artificial Sequence
  Synthetic polynucleotide
caggtgatgggagggttttgatgattttc

SEQ ID NO: 11 - DNA, 30 bp, Artificial Sequence
  Synthetic polynucleotide
gtttgtactctgacccttcttattcctctc

SEQ ID NO: 12 - DNA, 31 bp, Artificial Sequence; identical to SEQ ID NO: 8
  Synthetic polynucleotide

SEQ ID NO: 13 - DNA, 22 bp, Artificial Sequence
  Synthetic polynucleotide
actggaggatgggagactgtgt

SEQ ID NO: 14 - DNA, 23 bp, Artificial Sequence
  Synthetic polynucleotide
tcaggagtcgatctgcaaggtct

SEQ ID NO: 15 - DNA, 23 bp, Artificial Sequence
  Synthetic polynucleotide
tctgttgcaatggacaagtttgg

SEQ ID NO: 16 - DNA, 23 bp, Artificial Sequence
  Synthetic polynucleotide
cctccccagccccaataattatt